SEQUENCE LISTING (11 sequences)

SEQ ID NO: 1 - DNA, 47 bp, Artificial
  shRNA TIP5-1
ggacgataaagcaaagatgttcaagagacatctttgctttatcgtcc

SEQ ID NO: 2 - DNA, 47 bp, Artificial
  shRNA TIP5-2
gcagcccagggaaactagattcaagagatctagtttccctgggctgc

SEQ ID NO: 3 - DNA, 60 bp, Artificial
  miRNA TIP5-1
gatcagccgcaaactcctctgagttttggccactgactgactcagaggattgcggctgat

SEQ ID NO: 4 - DNA, 60 bp, Artificial
  miRNA TIP5-2
gcaaagatgggatcagttaagggttttggccactgactgacccttaacttcccatctttg

SEQ ID NO: 5 - DNA, 20 bp, Artificial
  Primer -168/-149 forward
gaccagttgttgctttgatg

SEQ ID NO: 6 - DNA, 20 bp, Artificial
  Primer -10/+10 reverse
gcgtgtcagtacctatctgc

SEQ ID NO: 7 - DNA, 19 bp, Artificial
  Primer -100/-84 forward
tcccgacttccagaatttc

SEQ ID NO: 8 - RNA, 47 nt, Artificial
  shRNA TIP5-1.1
ggacgauaaagcaaagauguucaagagacaucuuugcuuuaucgucc

SEQ ID NO: 9 - RNA, 47 nt, Artificial
  shRNA TIP5-2.1
gcagcccagggaaacuagauucaagagaucuaguuucccugggcugc

SEQ ID NO: 10 - RNA, 60 nt, Artificial
  miRNA TIP5-1.1
gaucagccgcaaacuccucugaguuuuggccacugacugacucagaggauugcggcugau

SEQ ID NO: 11 - RNA, 60 nt, Artificial
  miRNA TIP5-2.1
gcaaagaugggaucaguuaaggguuuuggccacugacugacccuuaacuucccaucuuug

(19) United States
(12) Patent Application Publication
(10) Pub. No.: US 2013/0210074 A1
(43) Pub. Date: Aug. 15, 2013

(54) EPIGENETIC ENGINEERING

(71) Applicants: Lore FLORIN, Vienna (AT); Barbara ENENKEL, Warthausen (DE); Martin FUSSENEGGER, Maegenwil (CH); Hitto KAUFMANN, Ulm (DE); Raffaella SANTORO, Zurich (CH)

(72) Inventors: Lore FLORIN, Vienna (AT); Barbara ENENKEL, Warthausen (DE); Martin FUSSENEGGER, Maegenwil (CH); Hitto KAUFMANN, Ulm (DE); Raffaella SANTORO, Zurich (CH)

(73) Assignee: BOEHRINGER INGELHEIM INTERNATIONAL GMBH, Ingelheim am Rhein (DE)

(21) Appl. No.: 13/859,097

(22) Filed: Apr. 9, 2013

Related U.S. Application Data
(63) Continuation of application No. 12/779,356, filed on May 13, 2010, now abandoned.

(30) Foreign Application Priority Data
May 15, 2009 (EP) 09 160 340.7

(51) Int. Cl. C12N 15/113; C12N 15/85; C12P 21/00

(52) U.S. Cl. CPC C12N 15/1136; C12P 21/00; C12N 15/85
USPC 435/69.1; 435/375; 435/358; 435/320.1

(57) ABSTRACT

The invention concerns the field of cell culture technology. It concerns production host cell lines with increased expression of ribosomal RNA (rRNA) achieved through reducing expression of NoCR proteins, especially of TIP-5. Those cell lines have improved secretion and growth characteristics in comparison to control cell lines.

The invention further concerns a method of producing proteins using the cells generated by the described method.

### BACKGROUND OF THE INVENTION

1. Technical Field

The invention concerns the field of cell culture technology. It concerns production host cell lines with increased expression of ribosomal RNA (rRNA) achieved through reducing expression of NoCR proteins, especially of TIP-5. Those cell lines have improved secretion and growth characteristics in comparison to control cell lines.

2. Background

Selection of mammalian high-producer cell lines remains a major challenge for the biopharmaceutical manufacturing industry.

On the way from DNA to product translation is a major bottleneck which can limit the specific productivity of mammalian production cell lines. Cells are able to upregulate the rate of protein synthesis either by increasing the translational efficiency of existing ribosomes or by increasing the capacity of translation through the production of new ribosomes (ribosome biogenesis). With about 80% of total nuclear transcription being dedicated to the synthesis of ribosomal RNA (rRNA), ribosome biogenesis is one of the major metabolic activities of mammalian cells. Ribosome assembly occurs within the nucleolus and requires coordinated expression of four rRNAs (45S pre-rRNA, which is subsequently processed into 18S, 5.8S, 28S and 5S rRNA) and about 80 ribosomal proteins (r-proteins). 45S pre-rRNA is transcribed in the nucleolus by polymerase I (Pol I), 5S RNA is transcribed by Pol III at the nucleolar periphery and then imported into the nucleolus and r-proteins are transcribed by Pol II. Thus, ribosome biogenesis requires orchestration of transcription by different polymerases operating in different compartments. In mammalian cells, these processes are largely unknown (Santoro, R. and Grummt, I (2001). Molecular mechanisms mediating methylation-dependent silencing of ribosomal gene transcription. Mol Cell 8, 719-725).

Transcription of 45S pre-rRNA is the key step of ribosome biogenesis. Mammalian haploid genomes contain about 200 ribosomal RNA genes of which only a fraction is transcribed at any given time, while the rest remains silent (Santoro, R., Li, J., and Grummt, I (2002). The nucleolar remodeling complex NoRC mediates heterochromatin formation and silencing of ribosomal gene transcription. Nat. Genet. 32, 393-396). Active and silent genes are distinct with respect to chromatin configuration: active genes have a euchromatic structure, whereas silent genes are heterochromatic. The promoter of active rRNA genes is free of CpG methylation and is associated with acetylated histones. The opposite is true of silent genes.

The presence of transcriptionally silent rRNA genes represents a limiting factor for the synthesis of rRNA and the production of ribosomes. It has been hypothesized that cells can modulate rDNA transcription levels by altering the transcriptional activity of each gene and/or by altering the number of active genes. However, a satisfying correlation between 45S pre-rRNA synthesis levels and the number of rRNA genes has not been found. For instance, in S. cerevisiae, reducing the number of rRNA genes by about two thirds did not affect total rRNA production. Similarly, maize inbred lines and aneuploid chicken cells, containing different numbers of rRNA copies displayed the same levels of rRNA transcription.

As rDNA represents the major component of the ribosome, silencing of these genes results in a limitation in ribosome biogenesis and thereby protein translation, thus ultimately leading to reduced protein synthesis.

In biopharmaceutical production cells, this creates a limit in the cell's full production capacity, meaning reduced specific productivities of the therapeutic protein product. It will thereby lead to reduced overall protein yields in industrial production processes.

The other factor next to the specific productivity (P_{spec}) determining process yield (Y) is the IVC, the integral of viable cells over time which produce the desired protein. This correlation is expressed by the following formula: Y=P_{spec}*IVC. Therefore, there is an urgent need to increase either the production capacity of the host cell or viable cell densities in the bioreactor by improving cell growth—or ideally both parameters at the same time.

### SUMMARY OF THE INVENTION

The present invention solves the above described problem and shows that the knockdown of TIP-5, a subunit of NoRC (nucleolar remodeling complex; McStay, B. and Grummt, I. (2008). The epigenetics of rRNA genes: from molecular to chromosome biology. Annu. Rev Cell Dev. Biol 24, 131-157), decreases the number of silent rRNA genes, upregulates rRNA transcription, enhances ribosome synthesis and increases production of recombinant proteins.

The data of the present application demonstrate that the number of transcriptionally competent rRNA genes limits ribosome synthesis. Epigenetic engineering of ribosomal RNA genes offers new possibilities for improving biopharmaceutical manufacturing and provides novel insights into the complex regulatory network which governs the translation machinery.

The present application shows that knockdown of TIP-5 induces loss of repressive chromatin marks at the rDNA repeats, enhances rDNA transcription, alters nucleolus structure and promotes cell growth and proliferation.

To determine whether increasing numbers of active rRNA genes affect cellular growth and proliferation, we analyzed several shRNA-TIP5 cells by flow cytometry (FACS).

Surprisingly and for the first time, we show in the present application that an engineered decrease in the number of silent rRNA genes could be correlated with enhanced production of rRNA and ribosomes and consequently with higher productivity of mammalian cells.

Unexpectedly, the present application additionally provides data showing that knock-down of TIP-5 in different mammalian cell lines leads to faster cell cycle progression and increased cell proliferation.

This finding is in contrast to what is described in the prior art (WO2009/017670). TIP-5 has previously been identified to function as a Ras-mediated epigenetic silencing effector (RESE) for Fas in a global miRNA screen (WO2009/017670). Ras is a well known oncogene involved in cell transformation and tumorigenesis which is frequently mutated or overexpressed in human cancers. Therefore, the prior art claims that reduced expression on Ras effectors such as TIP-5 results in an inhibition of cell proliferation.

To verify this, we analyzed both shRNA-TIP5 cells by flow cytometry (FACS). As shown in FIG. 4A,B, however, the number of shRNA-TIP-5 cells in S-phase is significantly higher in shRNA-TIP5 cells in comparison to control cells. Consistent with these results, shRNA TIP5 cells showed increased incorporation of 5-bromodeoxyuridine (BrdU) into nascent DNA and higher levels of Cyclin A (FIG. 4C).

Additionally, we compared cell proliferation rates between shRNA-TIP5, shRNA-control and parental NIH3T3 and CHO-K1 cells (FIG. 4D,F). Surprisingly and in contrast to prior art reports, both NIH/3T3 and CHO-K1 cells, expressing miRNA-TIP5 sequences, proliferate at a faster rate than the control cells. Thus, a decrease in the number of silent rRNA genes does have an impact on cell metabolism. The present invention surprisingly shows that depletion of TIP5 and a consequent decrease in rDNA silencing enhances cell proliferation.

The present application demonstrates a significant increase in protein production in TIP5-depleted cells compared to the control cell lines (see Example 6, FIG. 6). The increase in protein production in TIP5-depleted cells compared to the control cell lines is more than 2-fold, more than 4-fold, more than 5-fold, more than 6-fold, more than 10-fold, between 2-10-fold. These data show that TIP5-depletion increases heterologous protein production. The present application shows that a decrease in the number of silent rRNA genes enhances ribosome synthesis and increases the potential of the cells to produce recombinant proteins.

In this invention, we provide a new method for increasing rRNA transcription, ribosome biogenesis and translation by reducing TIP-5 with the benefit to ultimately enhance secretion of recombinant proteins.

Furthermore, we demonstrate that depletion of TIP-5 leads to faster cell cycle progression and improved cell growth.

Enhanced cell growth has a profound impact on multiple aspects of the biopharmaceutical production process:
Shorter generation times of cells, which results in shortened time lines in cell line development. Generation times are preferably shorten than 24 hrs, preferably between 20 to 24 hrs, more preferably between 15 to 24 hrs or 15 to 22 hrs, most preferably between 10-24 hrs.
Higher efficiency after single-cell cloning and faster growth thereafter.
Shorter timeframes during scale-up, especially in the case of inoculum for a large-scale bioreactor.
Higher product yield per fermentation time due to the proportional correlation between IVC and product yield. Conversely, low IVCs cause lower yields and/or longer fermentation times. Preferably the yield is increased by 10%, more preferably by 20% most preferably by 30%.

This enables to increase the protein yield in production processes based on eukaryotic cells. It thereby reduces the cost of goods of such processes and at the same time reduces the number of batches that need to be produced to generate the material needed for research studies, diagnostics, clinical studies or market supply of a therapeutic protein. The invention furthermore speeds up drug development as often the generation of sufficient amounts of material for pre-clinical studies is a critical work package with regard to the timeline.

The invention can be used to increase the property of all eukaryotic cells used for the generation of one or several specific proteins for either diagnostic purposes, research purposes (target identification, lead identification, lead optimization) or manufacturing of therapeutic proteins either on the market or in clinical development.

The cell lines provided by this invention help to increase the protein yield in production processes based on eukaryotic cells. This reduces the cost of goods of such processes and at the same time it reduces the number of batches that need to be produced to generate the material needed for research studies, diagnostics, clinical studies or market supply of a therapeutic protein.

The invention furthermore speeds up drug development as often the generation of sufficient amounts of material for pre-clinical studies is a critical work package with regard to the timeline.

The optimized host cell lines with reduced expression of TIP-5 can be used for the generation of one or several specific proteins for either diagnostic purposes, research purposes (target identification, lead identification, lead optimization) or manufacturing of therapeutic proteins either on the market or in clinical development.

They are equally applicable to express or produce secreted or membrane-bound proteins (such as surface receptors, GPCRs, metalloproteases or receptor kinases) which share the same secretory pathways and are equally transported in lipid-vesicles. The proteins can then be used for research purposes which aim to characterize the function of cell-surface receptors, e.g. for the production and subsequent purification, crystallization and/or analysis of surface proteins. This is of crucial importance for the development of new human drug therapies as cell-surface receptors are a predominant class of drug targets. Moreover, it might be advantageous for the study of intracellular signalling complexes associated with cell-surface receptors or the analysis of cell-cell-communication which is mediated in part by the interaction of soluble growth factors with their corresponding receptors on the same or another cell.

### DESCRIPTION OF THE FIGURES

FIG. 1: KNOCK-DOWN OF TIP-5 IN RODENT AND HUMAN CELL LINES

(A,B) qRT-PCR of TIP5 mRNA of (A) NIH/3T3 cells stably expressing shRNA-TIP5-1 and TIP5-2 sequences and (B) of HEK293T cells stably expressing miRNA-TIP5-1 and TIP5-2 sequences. Data were normalized to GAPDH mRNA levels.
(C) Semiquantitative RT-PCR of TIP5 mRNA of stable shRNA-TIP5-1/2 NIH/3T3, miRNA-TIP5-1/2 HEK293T and miRNA-TIP5-1/2 CHO-K1 cells. As control, qRT-PCR of GAPDH mRNA is shown.

FIG. 2: TIP-5 KNOCKDOWN LEADS TO REDUCED RDNA METHYLATION

(A-C) Depletion of TIP5 decreases CpG methylation of rDNA promoters. Upper panels: Diagrams of (A) mouse, (B) human and (C) Chinese hamster rDNA promoter regions including the HpaII (H) sites analyzed. Black circles indicate CpG dinucleotides. Arrows represent the primers used to amplify HpaII-digested DNA.
Lower panels: rDNA CpG methylation levels were measured in (A) NIH/3T3, (B) HEK293T and (C) CHO-K1 cells stably expressing shRNA- and/or miRNATIP5-1/2 and control sequences. Data represent the amounts of HpaII-resistant rDNA normalized to the total rDNA calculated by amplification with primers encompassing DNA sequences lacking HpaII-sites and undigested DNA.
(D,E) Depletion of TIP5 decreases rDNA CpG methylation levels. Analysed is (A) the rDNA intergenic and promotor region including the transcription start site (+1) and (B) two areas within the coding region. Schema representing a single mouse rDNA repeat and the analyzed HpaII (H) sites. Arrows represent the primers used to amplify HpaII digested DNA. Data represent the amounts of HpaII resistant rDNA normalized to the total rDNA calculated by amplification with primers encompassing DNA sequences lacking HpaII sites and undigested DNA.

FIG. 3: INCREASED RRNA LEVELS IN TIP-5 KNOCKDOWN CELLS

(A) Depletion of TIP5 enhances rRNA synthesis. qRT-PCR-based 45S pre-rRNA levels of stable NIH/3T3 and HEK293T cell lines were normalized to GAPDH mRNA levels.
(B) rDNA transcription was detected by in situ BrUTP incorporation after same exposure time. The BrUTP signal (left panel) is higher in TIP-5 depleted cells and is specifically detected in the nucleolus (darker areas within the nucleus as seen in the phase contrast images (right panel).

FIG. 4: TIP-5 DEPLETION LEADS TO INCREASED PROLIFERATION AND CELL GROWTH

(A) FACS analysis of shRNA TIP5 cells
(B) Percentage of cells in individual cell cycle phases. The number or percentage of cells in S phase increases, whereas the number or percentage of cells in G1 phase decreases in TIP5 depleted cells. Proliferation is enhanced.
(C) BrdU incorporation assay. Cells were incubated with 10 μM BrdU for 30 min, stained with antibodies to BrdU, and percentage of cells in S phase was estimated. The BrdU assay shows increased DNA synthesis in TIP5 cells.
(D-F) Growth curves of (D) NIH/3T3, (E) HEK293T and (F) CHO-K1 cells stably expressing miRNA-TIP5 and control sequences. The growth curves demonstrate that TIP-5 depelted cells grow at least as fast as (HEK293) or even faster than control cells (NIH3T3 and CHO-K1).

FIG. 5: RIBOSOME ANALYSIS IN TIP-5 KNOCKDOWN CELLS

(A-C) Relative amounts of cytoplasmic RNA/cell in (A) stable NIH/3T3, (B) HEK293T and (C) CHO-K1 cells. Data represent the average of two experiments performed in triplicate.
(D) Ribosome profile of stable HEK293T and
(E) CHO-K1 cell lines.
More ribosomes are present in TIP5 knockdown cells.

FIG. 6: TIP-5 KNOCKDOWN LEADS TO ENHANCED PRODUCTION OF REPORTER PROTEINS

(A-C) SEAP expression of (A) stable NIH/3T3, (B) HEK293T and (C) CHO-K1 cell lines engineered with the constitutive SEAP expression vector pCAG-SEAP.
(D,E) Luciferase expression of (D) stable NIH/3T3 and (E) HEK293T cell lines engineered with the constitutive luciferase expression vector pCMV-luciferase.

### DETAILED DESCRIPTION OF THE INVENTION

### Knock-Down of TIP-5:

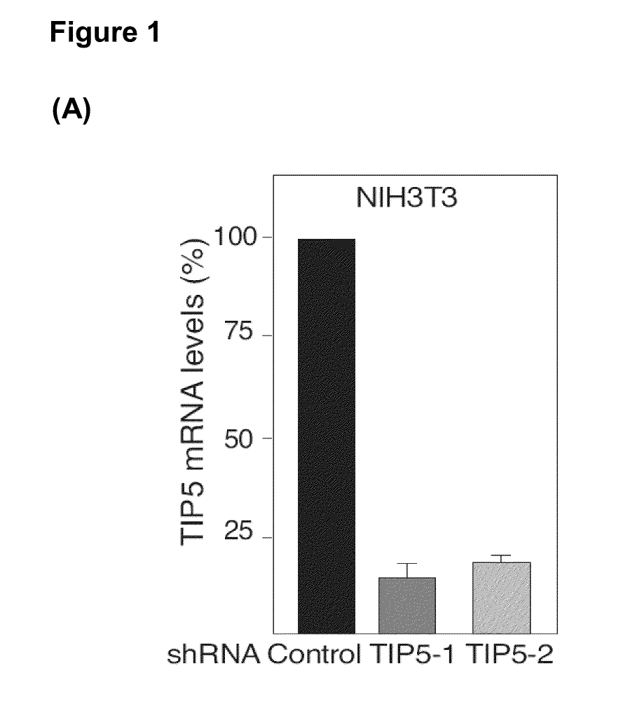

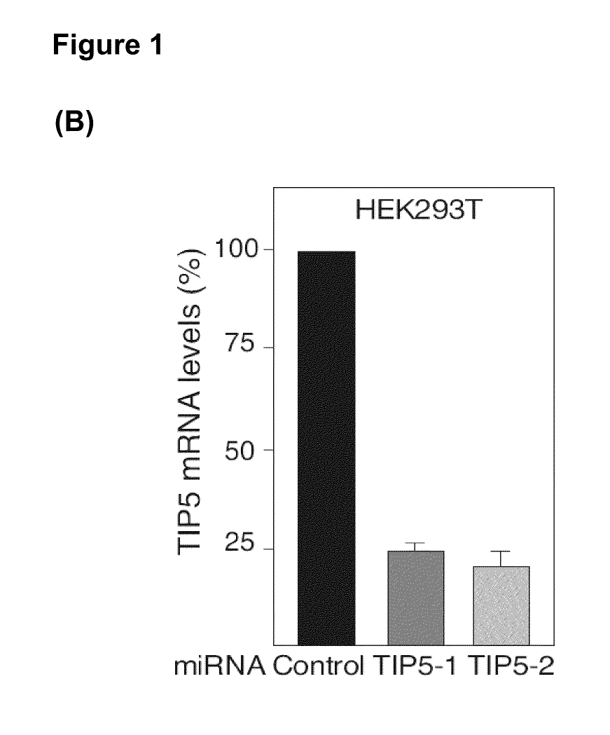

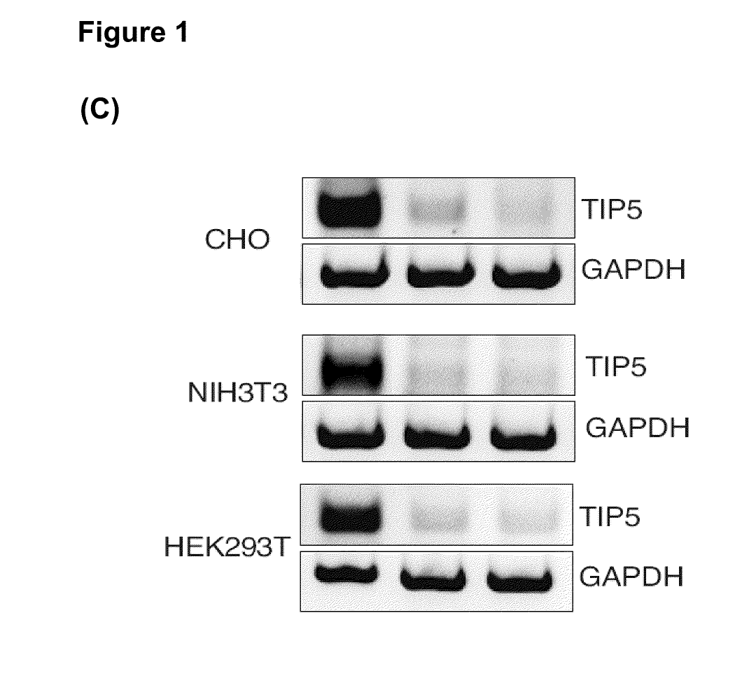

With the aim of engineering cells for increased synthesis of recombinant proteins, we determine whether a decrease in the number of silent rRNA genes enhances 45S pre-rRNA synthesis and, as consequence, also stimulates ribosome biogenesis and increases the number of translation-competent ribosomes. Therefore, we use RNA interference to knock down TIP5 expression and constructed stably transgenic shRNAexpressing NIH/3T3 or miRNA-expressing HEK293T and CHO-K1 using shRNA/miRNA sequences specific for two different regions of TIP5 (TIP5-1 and TIP5-2). Stable cell lines expressing scrambled shRNA and miRNA sequences were used as control. There are two reasons for producing stable cell lines rather than performing transient transfections with plasmids expressing shRNA-TIP5 or miRNA-TIP5 sequences. First, the loss of repressive epigenetic marks like CpG methylation is a passive mechanism, requiring multiple cell divisions. Second, even though HEK293T cells can be transfected relatively easily, the poor transfection efficiency of NIH/3T3 and CHO-K1 cells would compromise subsequent analyses of endogenous rRNA, ribosome levels and cell growth properties. To determine the efficiency of TIP5 knockdown in the selected clones, we measure TIP5 mRNA levels by quantitative and semiquantitative reverse-transcriptase-mediated PCR (FIG. 1). TIP5 expression decreases about 70-80% in NIH/3T3/shRNA-TIP5-1 and -2 cells when compared to control cells (FIG. 1A). A similar reduction in TIP5 mRNA levels is observed in stable HEK293T (FIG. 1B). TIP5 mRNA levels in CHO-K1-derived cells could be measured only by semiquantitative PCR (FIG. 1C) but the reduction of TIP5 mRNA was similar to that of stable NIH/3T3 and HEK293T cells. These results demonstrate that the established cell lines contain low levels of TIP5.

TIP-5 Knockdown Leads to Reduced rDNA Methylation:

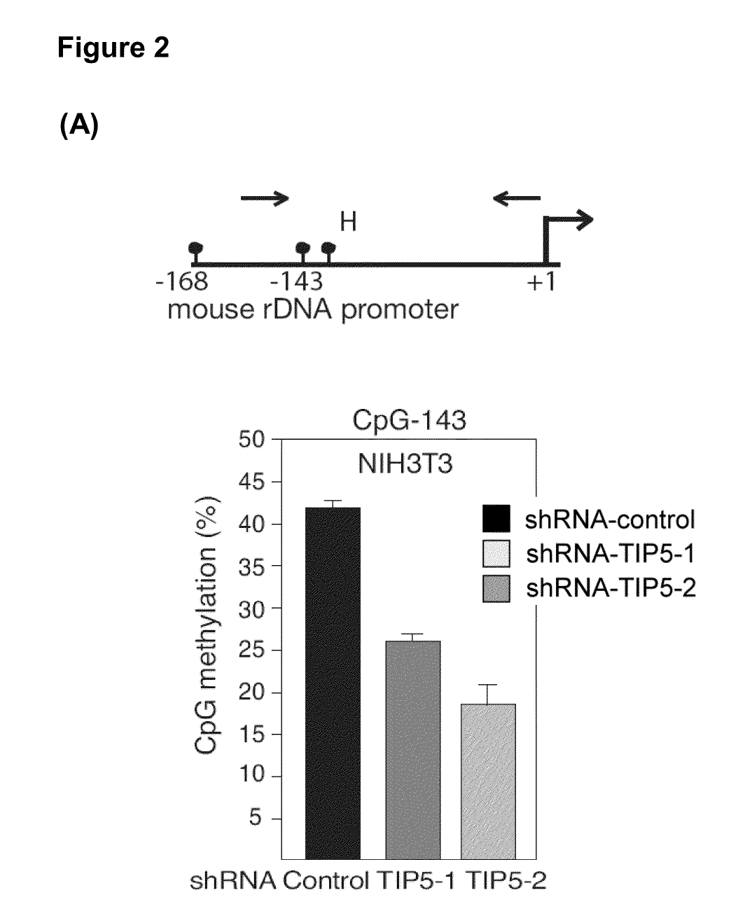

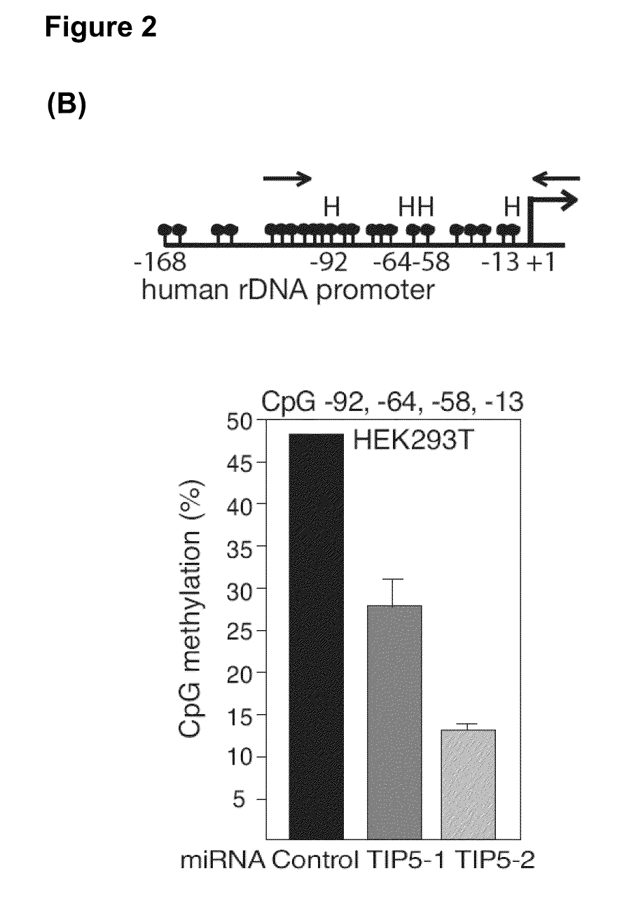

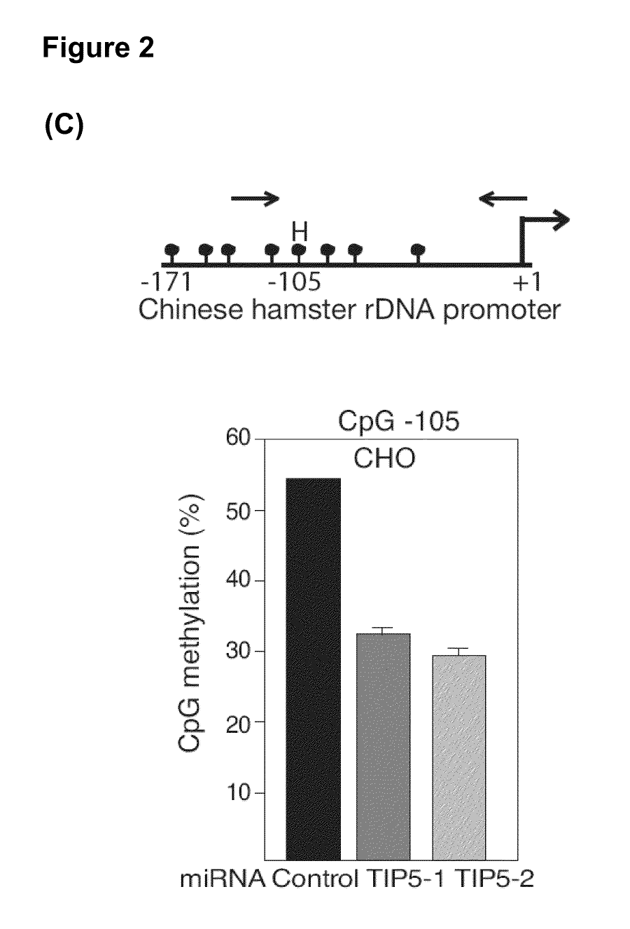

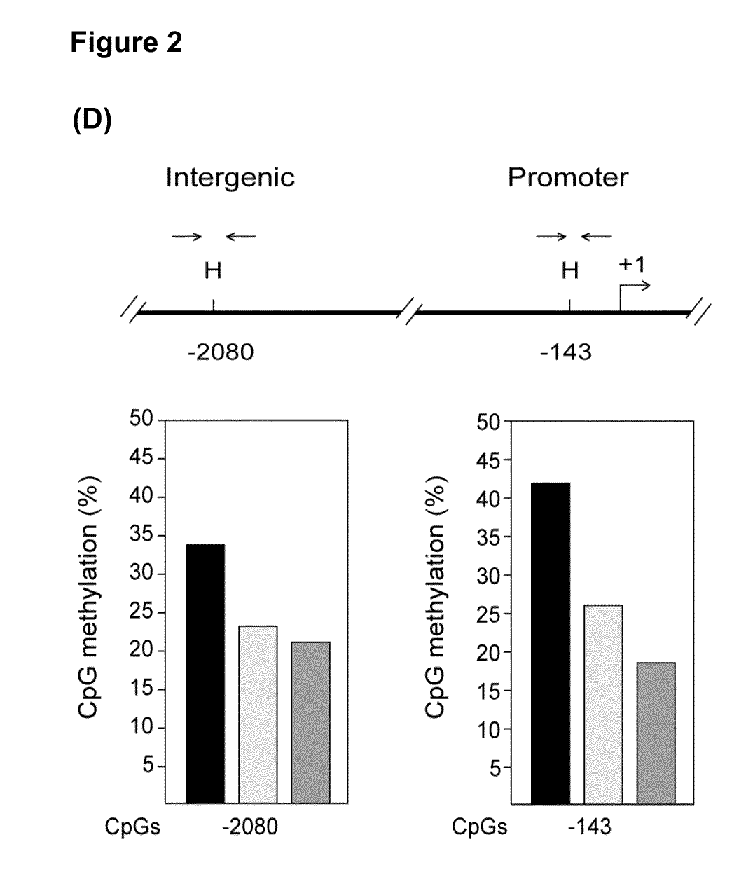

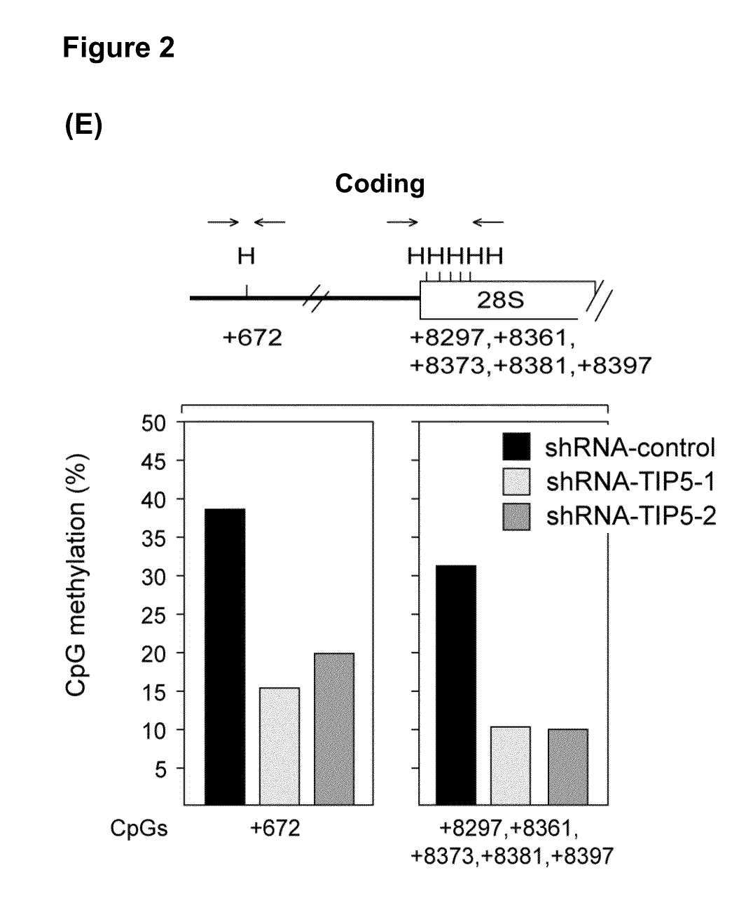

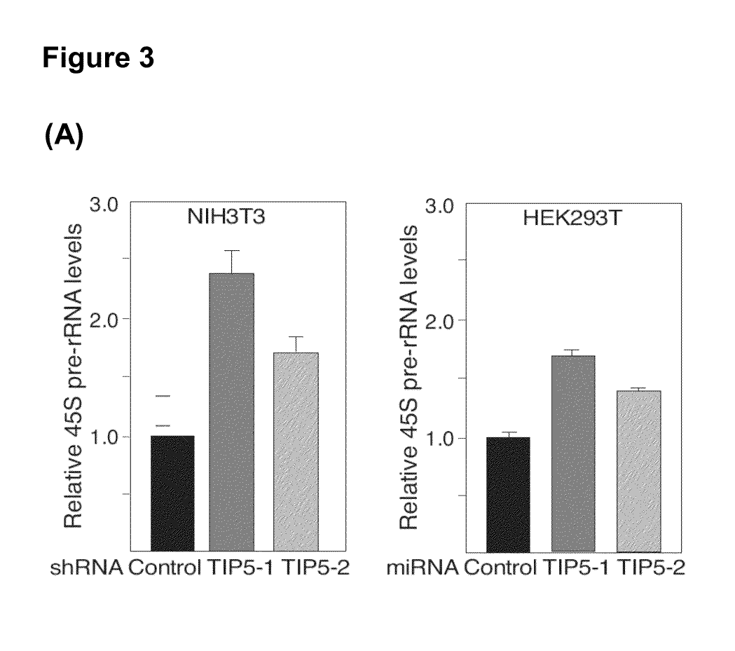

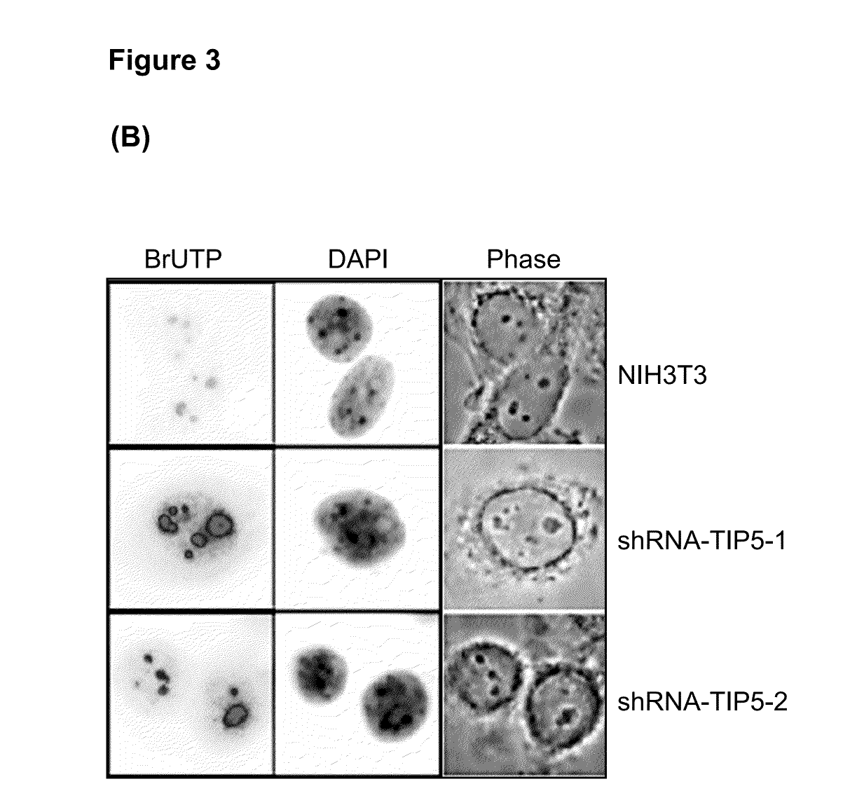

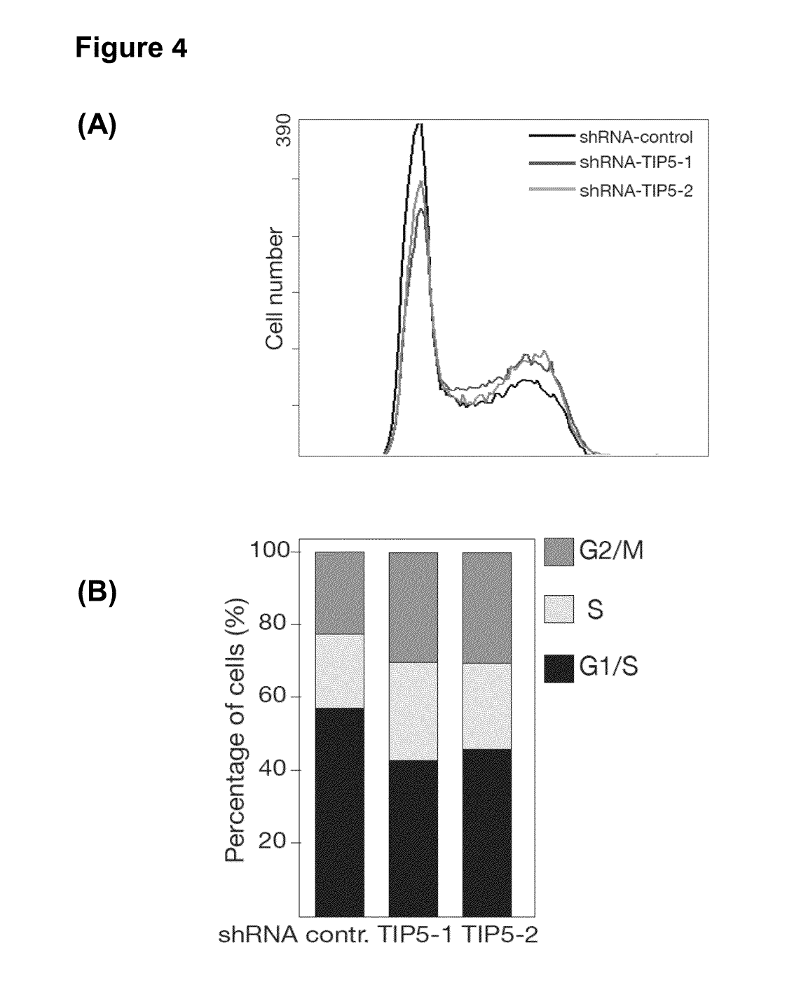

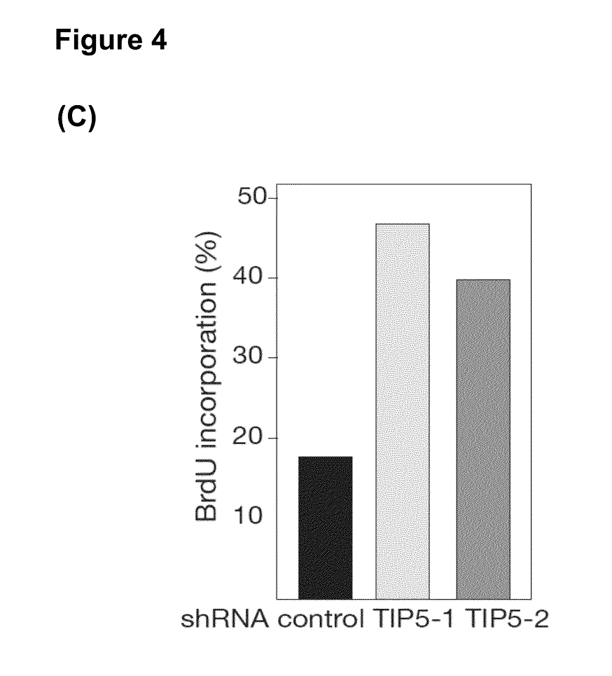

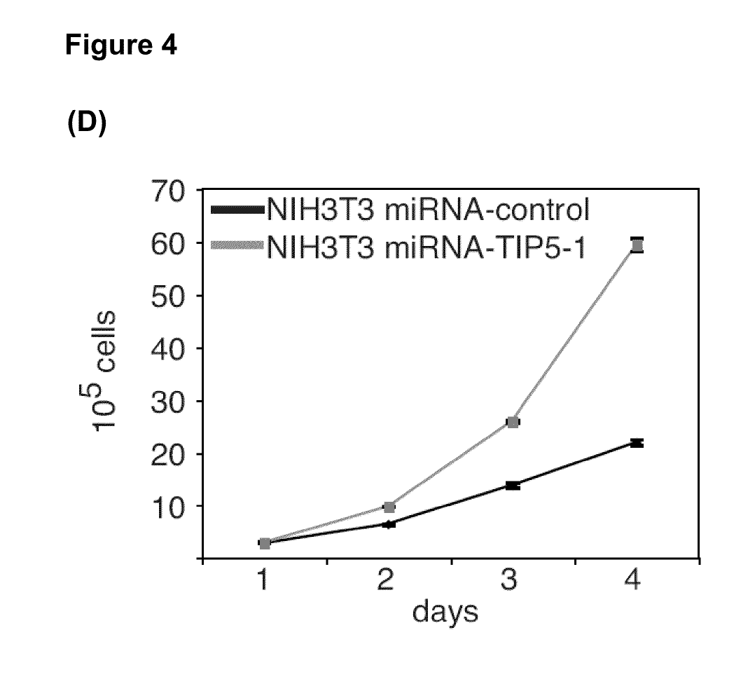

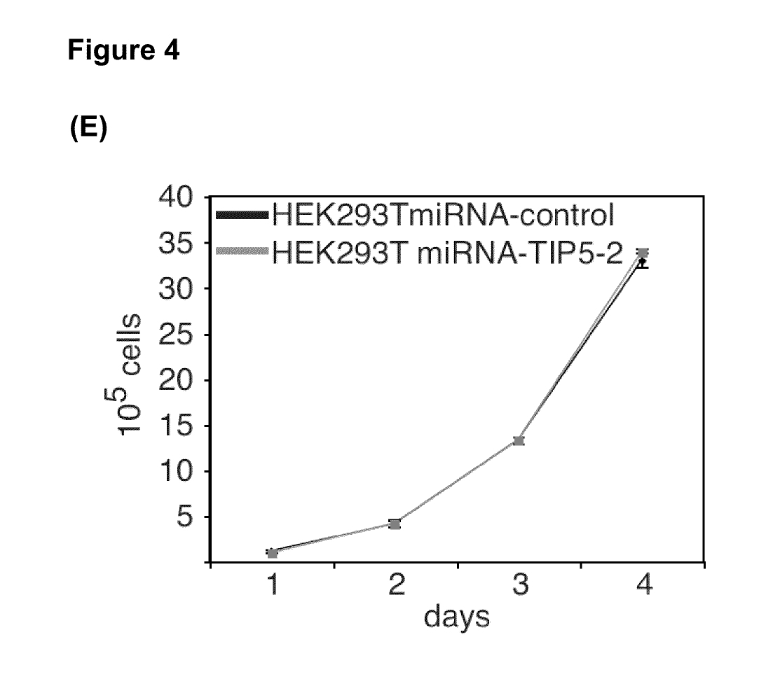

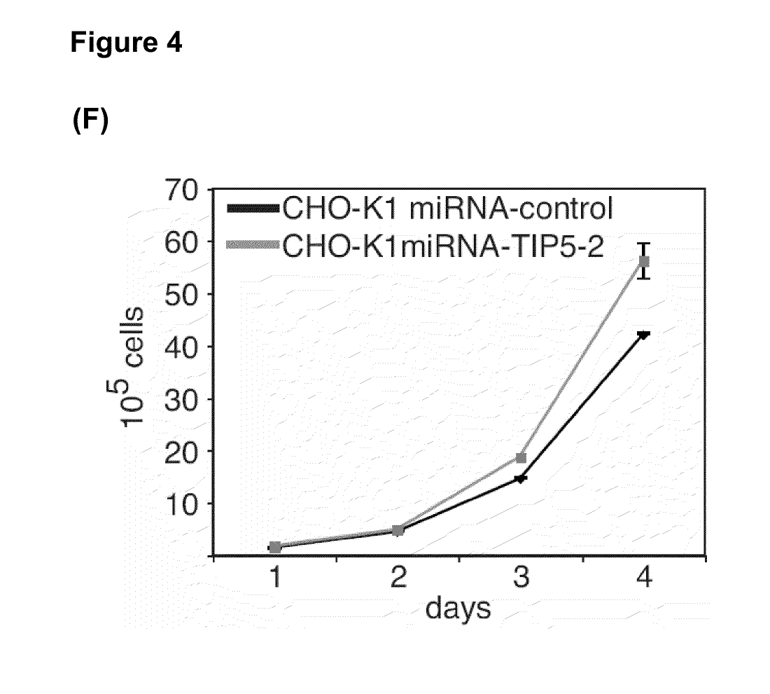

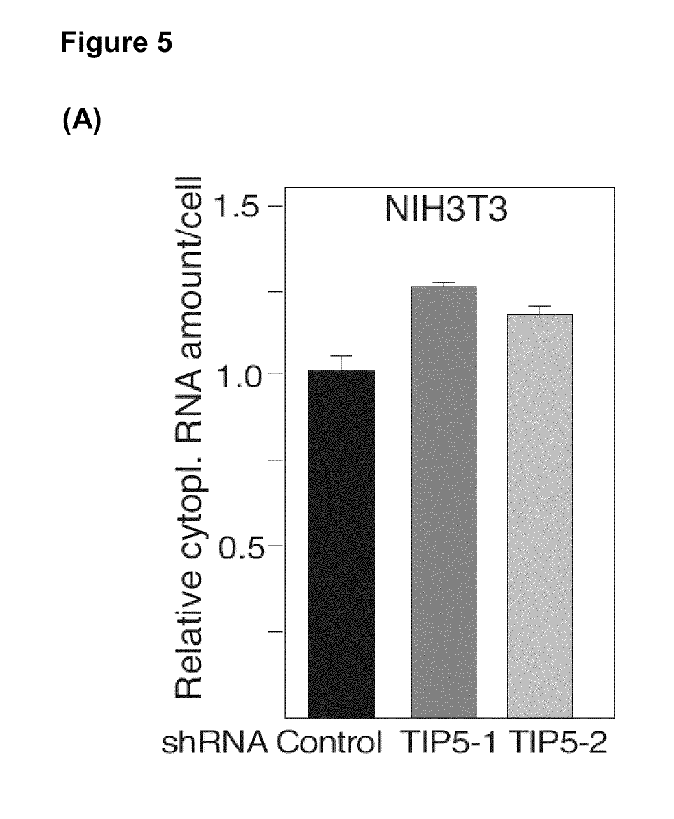

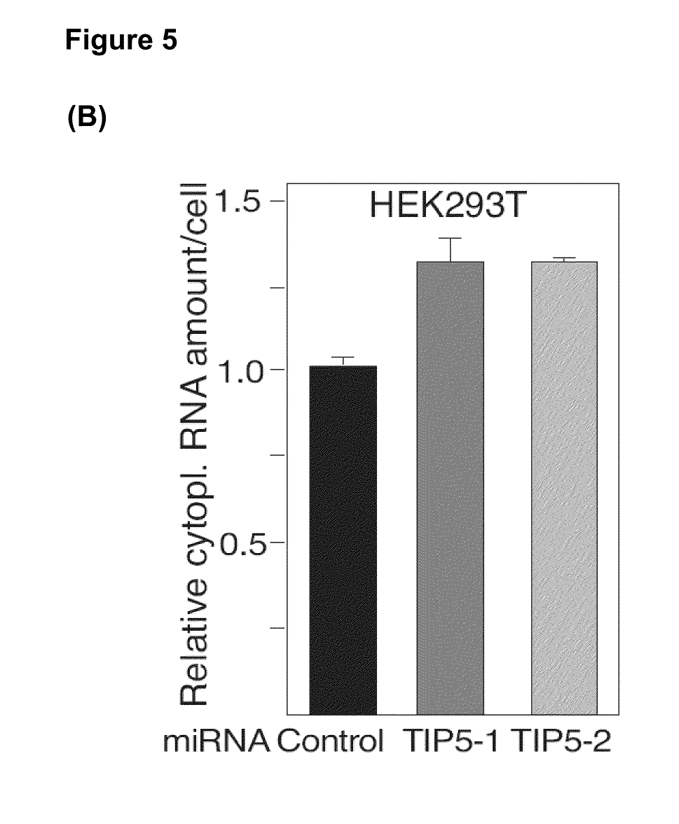

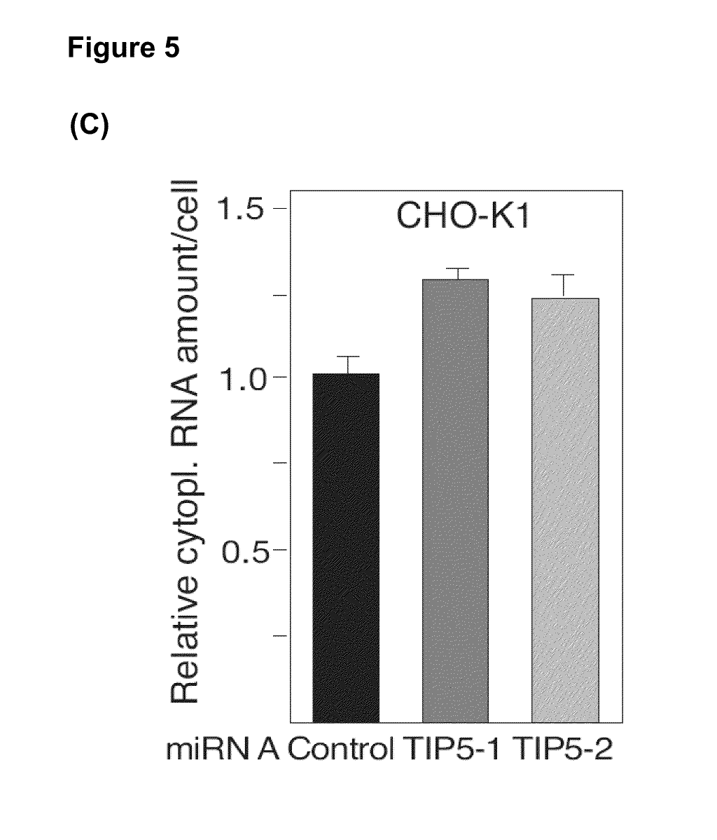

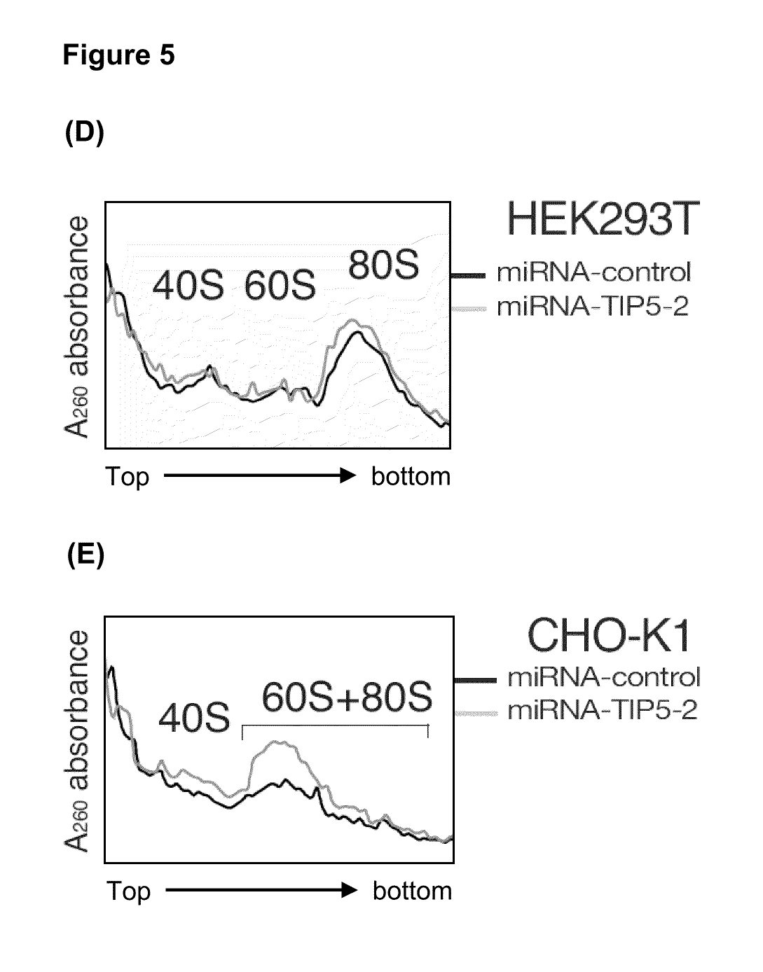

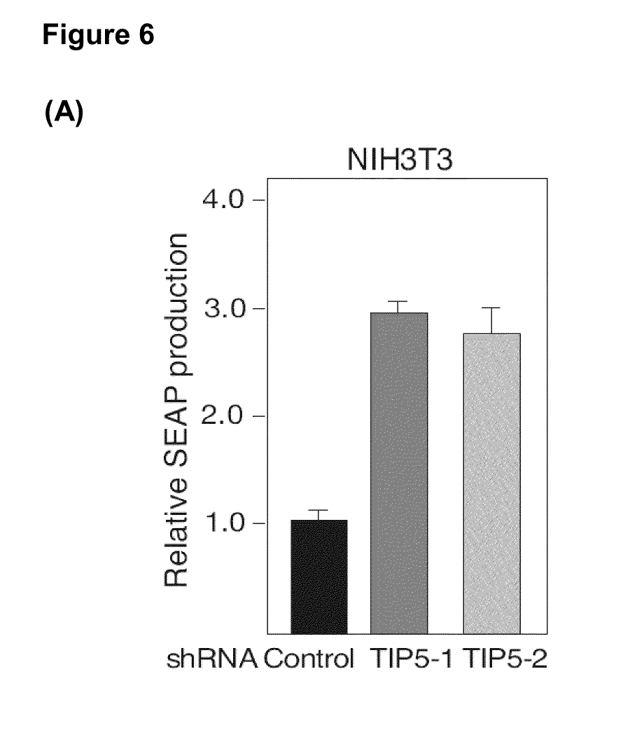

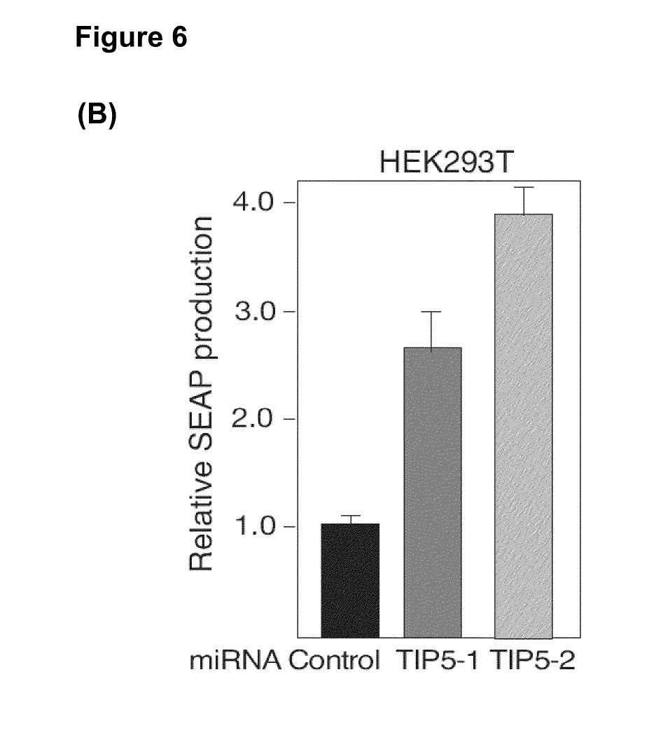

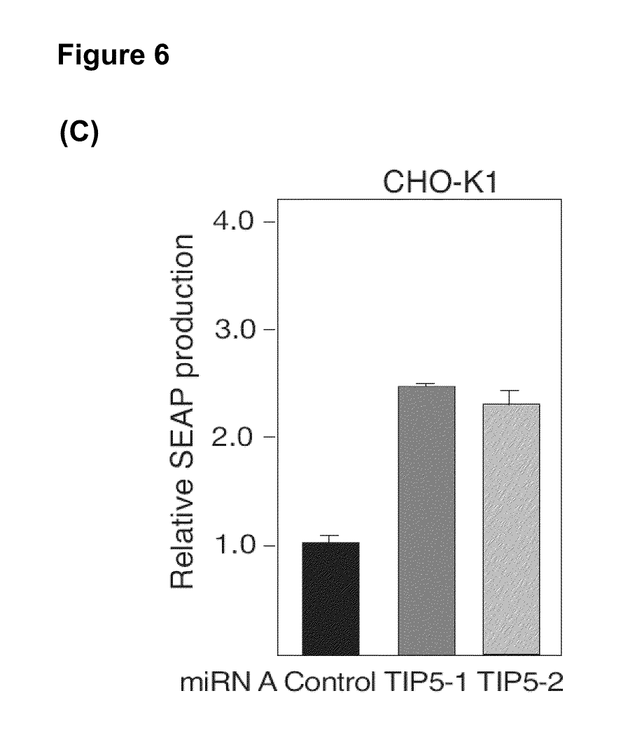

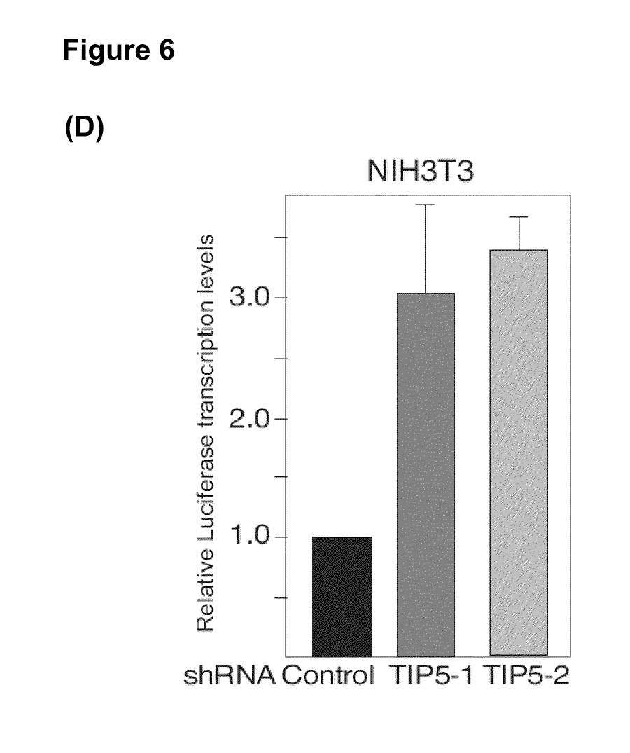

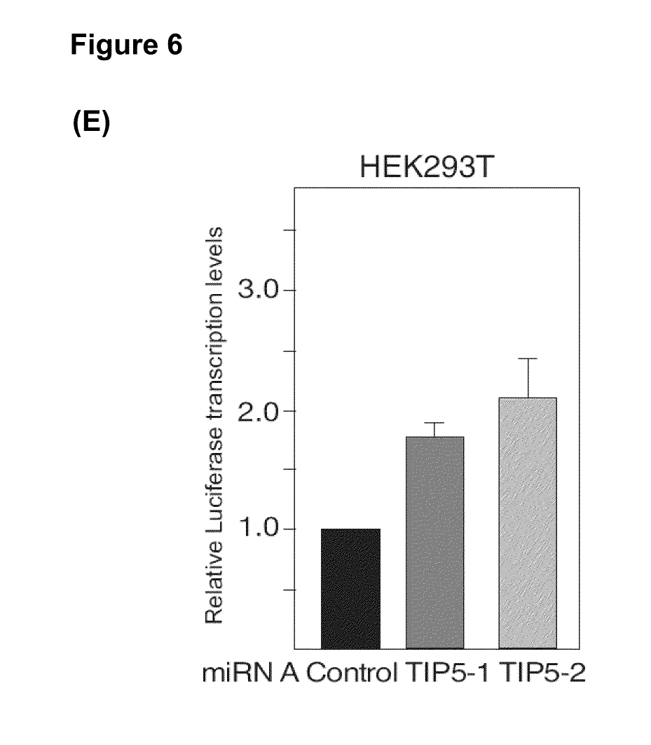

CpG methylation of the mouse rDNA promoter impairs binding of the basal transcription factor UBF, and the formation of preinitiation complexes is prevented (Sanij, E., Poortinga, G., Sharkey, K., Hung, S., Holloway, T. P., Quin, J., Robb, E., Wong, L. H., Thomas, W. G., Stefanovsky, V., Mos s, T., Rothblum, L., Hannan, K. M., McArthur, G. A., Pearson, R. B., and Hannan, R. D. (2008). UBF levels determine the number of active ribosomal RNA genes in mammals. J. Cell Biol 183, 1259-1274). In NIH/3T3 cells about 40% to 50% of rRNA genes contain CpG-methylated sequences and are transcriptionally silent. The sequences and CpG density of the rDNA promoter in humans, mice and Chinese hamsters differ significantly. In humans, the rDNA promoter contains 23 CpGs, while in mice and Chinese hamsters there are 3 and 8 CpGs, respectively (FIG. 2A-C). To verify that TIP5 knockdown affects rDNA silencing, we determine the rDNA methylation levels by measuring the amount of meCpGs in the CCGG sequences. Genomic DNA is HpaII-digested, and resistance to digestion (i.e. CpG methylation) is measured by quantitative real-time PCR using primers encompassing HpaII sequences (CCGG). There is a decrease in CpG methylation within the promoter region of a the majority of rRNA genes in all TIP5 knock-down cell lines, underscoring the key role of TIP5 in promoting rDNA silencing (FIG. 2).

Notably, although TIP5 binding and de novo methylation is restricted to the rDNA promoter sequences, CpG methylation amounts in TIP-5 reduced NIH3T3 cells diminished over the entire rDNA gene (intergenic, promoter and coding regions; FIG. 2D,E), indicating that TIP5, once bound to the rDNA promoter, initiates spreading mechanisms for the establishment of silent epigenetic marks throughout the rDNA locus.

Increased rRNA Levels in Tip-5 Knockdown Cells:

To determine whether a decrease in the number of silent genes affects the amounts of the rRNA transcript, we measure 45S pre-rRNA synthesis by qRT-PCR using primers that encompassed the first rRNA processing site (FIG. 3A) and by in vivo BrUTP incorporation (FIG. 3B). As expected, in both TIP5-depleted NIH/3T3 and HEK293T cells, an enhancement of rRNA production compared to the control cell line is detected by both analyses

### TIP-5 Depletion Leads to Increased Proliferation and Cell Growth:

Ras is a well known oncogene involved in cell transformation and tumorigenesis which is frequently mutated or overexpressed in human cancers. Green et al. in WO2009/017670 describe to have identified TIP-5 to function as a Ras-mediated epigenetic silencing effector (RESE) of Fas in a global miRNA screen. The publication describes that reduced expression of Ras effectors such as TIP-5 results in an inhibition of cell proliferation. We analyze both shRNA-TIP5 cells by flow cytometry (FACS). As shown in FIGS. 4A,B, the numbers of cells in S-phase were significantly higher in both shRNA-TIP5 cells in comparison to control cells. A similar profile was obtained with NIH3T3 cells 10 days after infection with a retrovirus expressing miRNA directed against TIP5 sequences. Consistent with these results, shRNA TIP5 cells show increased incorporation of 5-bromodeoxyuridine (BrdU) into nascent DNA and higher levels of Cyclin A (FIG. 4C). Finally, we compare cell proliferation rates between shRNA-TIP5, shRNA-control and parental NIH3T3, HEK293 and CHO-K1 cells (FIG. 4D-F). Surprisingly and in contrast to the prior art reports, both NIH/3T3 and CHO-K1 cells, expressing miRNA-TIP5 sequences, proliferate at faster rates than the control cells, suggesting that a decrease in the number of silent rRNA genes does have an impact on cell metabolism. TIP5 depletion in HEK293T did not significantly affect cell proliferation, because these cells had already reached their maximum rate of proliferation. These data surprisingly show that depletion of TIP5 and a consequent decrease in rDNA silencing enhance cell proliferation.

### Ribosome Analysis in TIP-5 Knockdown Cells:

In mammalian cell cultures, the rate of protein synthesis is an important parameter, which is directly related to the product yield. To determine whether depletion of TIP5 and a consequent decrease in rDNA silencing increases the number of translation-competent ribosomes in the cell, we initially measure the levels of cytoplasmic rRNA. In the cytoplasm, most of the RNA consists of processed rRNAs assembled into ribosomes. As shown in FIG. 5A-C, all TIP5-depleted cell lines containe more cytoplasmic RNA per cell, suggesting that these cells produce more ribosomes. Also, analysis of the polysome profile shows that TIP5depleted HEK293 and CHO-K1 cells contained more ribosome subunits (40S, 60S and 80S) compared to control cells (FIG. 5D).

### Tip-5 Knockdown Leads to Enhanced Production of Reporter Proteins:

To determine whether depletion of TIP5 and decrease in rDNA silencing enhance heterologous protein production, we transfect stable TIP5-depleted NIH/3T3, HEK293T and CHO-K1 derivatives with expression vector promoting constitutive expression of the human placental secreted alkaline phosphatase SEAP (pCAG-SEAP; FIG. 6A-C) or luciferase (pCMV-luciferase; (FIG. 6D,E). Quantification of protein production after 48 h reveals a two- to four-fold increase in both SEAP and luciferase production in TIP5-depleted cells compared to the control cell lines, indicating that TIP5-depletion increases heterologous protein production. All these results show that a decrease in the number of silent rRNA genes enhances ribosome synthesis and increases the potential of the cells to produce recombinant proteins.

### TIP-5 Knockout Increases Biopharmaceutical Production of Monocyte Chemoattractant Protein 1 (MCP-1) and Enhances Therapeutic Antibody Production:

(a) A CHO cell line (CHO DG44) secreting monocyte chemoattractant protein 1 (MCP-1) or a therapeutic antibody is transfected with an empty vector (MOCK control) or small RNAs (shRNA or RNAi) designed to knock-down TIP-5 expression. The highest MCP-1 titers are seen in the cell pools with the most efficient TIP-5 depletion, whereas the protein concentrations are markedly lower in mock transfected cells or the parental cell line.
b) CHO host cells (CHO DG44) are first transfected with short RNAs sequences (shRNAs or RNAi) to reduce TIP-5 expression and stable TIP-5 depleted host cell lines are generated. Subsequently these cell lines and in parallel CHO DG 44 wild type cells are transfected with a vector encoding monocyte chemoattractant protein 1 (MCP-1) or a therapeutic antibody as the gene of interest. The highest MCP-1 titers and productivities are seen in the cell pools with the most efficient TIP-5 depletion, whereas the protein concentrations are markedly lower in mock transfected cells or the parental cell line.
c) When the same cells described in a) or b) are subjected to batch or fed-batch fermentations, the differences in overall MCP-1 titers or antibody titers are even more pronounced: As the cells transfected with reduced expression of TIP-5 grow faster and also produce more protein per cell and time, they exhibit higher IVCs and show higher productivities at the same time. Both properties have a positive influence on the overall process yield. Therefore, Tip5 deleted cells have significantly higher MCP-1 or antibody harvest titers and lead to more efficient production processes.

Also SNF2H deleted cells have significantly higher IgG harvest titers and lead to more efficient production processes.

Knock-Out of the TIP-5 Gene Increases rRNA Transcription and Enhances Proliferation Most Efficiently:

The most efficient way to generate an improved production host cell line with constantly reduced levels of TIP-5 expression is to generate a complete knock-out of the TIP-5 gene. For this purpose, one can either use homologous recombination or make use of the Zink-Finger Nuclease (ZFN) technology to disrupt the Tip-5 gene and prevent its expression. As homologous recombination is not efficient in CHO cells, we design a ZFN which introduces a double strand break within the TIP-5 gene which is thereby functionally destroyed. To control efficient knock-out of TIP-5, a Western Blot is performed using anti-TIP-5 antibodies. On the membrane, no TIP-5 expression is detected in TIP-5 knock-out cells wherease the parental CHO cell line shows a clear signal corresponding to the TIP-5 protein.

Next, rRNA transcription is analysed in TIP-5 knock-out CHO cells and the parental CHO cell line. The assay confirms higher levels of rRNA synthesis and increased ribosome numbers in TIP-5 knock-out cells compared to either the parental cell and also compared to cells with only reduced TIP-5 expression levels.

Moreover, cells deficient for TIP-5 proliferate faster and show higher cell numbers in fed-batch processes compared to TIP5 wild-type cells and cell lines in which TIP-5 expression was only reduced by introduction of interfering RNAs (such as shRNA or RNAi).

The general embodiments “comprising” or “comprised” encompass the more specific embodiment “consisting of”. Furthermore, singular and plural forms are not used in a limiting way.

Terms used in the course of this present invention have the following meaning. The term “epigenetic engineering” means influencing epigenetic modifications of the chromatin without affecting the nucleic acid sequence. Epigenetic modifications include changes in the methylation or acetylation of histones or DNA nucleotides as well as alkylations. In the present invention, “epigenetic engineering” primarily refers to engineering in DNA methylation.

“NoRC” (nucleolar remodeling complex) is the key determinant of rDNA silencing and it consists of TIP-5 (TTF-1-interacting protein 5) and the ATPase SNF2h. NoRC binds to the rDNA promoter of silent genes and represses rDNA transcription through histone-modifying and DNA-methylating activities.

“TIP-5” or “TIP5” (transcription termination factor 1 (TTF1)-interacting protein 5) is a nucleolar protein of more than 200 kD that serves to recruit histone deacetylase activity to the rDNA by interacting with DNA-methyl-transferases (DNMTs) and histone deacetylases (HDACs) and other chromatin modifying factors. Further synonyms are: BAZ2A, WALp3; FLJ13768; FLJ13780; FLJ45876; KIAA0314 and DKFZp781B109

“SNF2h” is a member of the SWI/SNF family of proteins and has helicase and ATPase activities. SNF2h is a component of the NoRC involved in nucleosome gliding to establish a closed heterochromatic chromatin state. The official name of SNF2h is SMARCA5 (for SWI/SNF related, matrix associated, actin dependent regulator of chromatin, subfamily a, member 5). Further aliases are ISWI; hISWI; hSNF2H and WCRF135.

The expression “Reducing ribosomal RNA gene (rDNA) silencing” means influencing methylation and/or acetylation of the DNA encoding ribosomal RNA or the chromatin in this specific region resulting in a de-repression of rRNA gene transcription. More specifically, in the present invention the term refers to the approach to reduce the methylation of rRNA genes resulting in better accessibility of the genes for transcription factors and thus leading to the synthesis of more rRNA from the respective genes. “rDNA silencing” herein specifically refers to silencing of rRNA genes. It does not include unspecific, genome-wide silencing mechanisms which are not mediated by the NoRC.

rDNA Silencing can be Measured/Monitored by the Following Assays:

Silencing of rDNA results in reduced transcription of rRNA which can be analysed by quantitative or semi-quantitative PCR (e.g. using oligonucleotide primers against 45S pre-RNA as described in Materials and Methods).

Methylation of the rDNA gene promoters can be analysed by digestion of the genomic DNA with methylation-sensitive restriction enzymes and subsequent southern blotting, resulting in different band patterns for methylated and un-methylated status.

Alternatively, methylation-induced rDNA silencing can also be quantified by digestion of genomic DNA within methylation-sinsitive restriction enzymes and subsequent qPCR using primers spanning the site of cleavage (as described in Materials and Methods and shown in FIG. 2).

The term “knock-down” or “depletion” in the context of gene expression as used herein refers to experimental approaches leading to reduced expression of a given gene compared to expression in a control cell. Knock-down of a gene can be achieved by various experimental means such as introducing nucleic acid molecules into the cell which hybridize with parts of the gene's mRNA leading to its degradation (e.g. shRNAs, RNAi, miRNAs) or altering the sequence of the gene in a way that leads to reduced transcription, reduced mRNA stability or diminished mRNA translation.

A complete inhibition of expression of a given gene is referred to as “knock-out”. Knock-out of a gene means that no functional transcripts are synthesized from said gene leading to a loss of function normally provided by this gene. Gene knock-out is achieved by altering the DNA sequence leading to disruption or deletion of the gene or its regulatory sequences. Knock-out technologies include the use of homologous recombination techniques to replace, interrupt or delete crucial parts or the entire gene sequence or the use of DNA-modifying enzymes such as zink-finger nucleases to introduce double strand breaks into DNA of the target gene.

### Assays to Monitor/Prove Knock-Down or Knock-Out of a Gene are Manifold:

For example, reduction/loss of mRNA transcribed from a selected gene can be quantitated by Northern blot hybridization, ribonuclease RNA protection, in situ hybridization to cellular RNA or by PCR. Reduced abundance/loss of the corresponding protein(s) encoded by a selected gene can be quantitated by various methods, e.g. by ELISA, by Western blotting, by radioimmunoassays, by immunoprecipitation, by assaying for the biological activity of the protein, by immunostaining of the protein followed by FACS analysis or by homogeneous time-resolved fluorescence (HTRF) assays.

The term “derivative” as used in the present invention means a polypeptide molecule or a nucleic acid molecule which is at least 70% identical in sequence with the original sequence or its complementary sequence. Preferably, the polypeptide molecule or nucleic acid molecule is at least 80% identical in sequence with the original sequence or its complementary sequence. More preferably, the polypeptide molecule or nucleic acid molecule is at least 90% identical in sequence with the original sequence or its complementary sequence. Most preferred is a polypeptide molecule or a nucleic acid molecule which is at least 95% identical in sequence with the original sequence or its complementary sequence and displays the same or a similar effect on secretion as the original sequence.

Sequence differences may be based on differences in homologous sequences from different organisms. They might also be based on targeted modification of sequences by substitution, insertion or deletion of one or more nucleotides or amino acids, preferably 1, 2, 3, 4, 5, 7, 8, 9 or 10. Deletion, insertion or substitution mutants may be generated using site specific mutagenesis and/or PCR-based mutagenesis techniques. Corresponding methods are described by (Lottspeich and Zorbas, 1998) in Chapter 36.1 with additional references.

“Host cells” in the meaning of the present invention are eukaryotic cells, preferably mammalian cells, most preferably rodent cells such as hamster cells. Preferred cells are BHK21, BHK TK⁻, CHO, CHO-K1, CHO-DUKX, CHO-DUKX B1, and CHO-DG44 cells or the derivatives/progenies of any of such cell line. Particularly preferred are CHO-DG44, CHO-DUKX, CHO-K1 and BHK21, and even more preferred CHO-DG44 and CHO-DUKX cells. Most preferred are CHO-DG44 cells. In a specific embodiment of the present invention host cells mean murine myeloma cells, preferably NS0 and Sp2/0 cells or the derivatives/progenies of any of such cell line. Examples of murine and hamster cells which can be used in the meaning of this invention are also summarized in Table 1. However, derivatives/progenies of those cells, other mammalian cells, including but not limited to human, mice, rat, monkey, and rodent cell lines, or eukaryotic cells, including but not limited to yeast, insect and plant cells, can also be used in the meaning of this invention, particularly for the production of biopharmaceutical proteins.

TABLE 1
Eukaryotic production cell lines

| CELL LINE | ORDER NUMBER |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (= CHO duk⁻ , CHO/dhfr⁻ ) | |
| CHO-DUKX B11 | ATCC CRL-9010 |
| CHO-DG44 | (Urlaub et al., 1983) |
| CHO Pro-5 | ATCC CRL-1781 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |

Host cells are most preferred, when being established, adapted, and completely cultivated under serum free conditions, and optionally in media which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invtirogen), serum-free CHO Medium (Sigma), and protein-free CHO Medium (Sigma) are exemplary appropriate nutrient solutions. Any of the media may be supplemented as necessary with a variety of compounds examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that would be known to those skilled in the art. In the present invention the use of serum-free medium is preferred, but media supplemented with a suitable amount of serum can also be used for the cultivation of host cells. For the growth and selection of genetically modified cells expressing the selectable gene a suitable selection agent is added to the culture medium.

The term “protein” is used interchangeably with amino acid residue sequences or polypeptide and refers to polymers of amino acids of any length. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with like properties.

The term “polypeptide” means a sequence with more than 10 amino acids and the term “peptide” means sequences up to 10 amino acids length.

The present invention is suitable to generate host cells for the production of biopharmaceutical polypeptides/proteins. The invention is particularly suitable for the high-yield expression of a large number of different genes of interest by cells showing an enhanced cell productivity.

“Gene of interest” (GOI), “selected sequence”, or “product gene” have the same meaning herein and refer to a polynucleotide sequence of any length that encodes a product of interest or “protein of interest”, also mentioned by the term “desired product”. The selected sequence can be full length or a truncated gene, a fusion or tagged gene, and can be a cDNA, a genomic DNA, or a DNA fragment, preferably, a cDNA. It can be the native sequence, i.e. naturally occurring form(s), or can be mutated or otherwise modified as desired. These modifications include codon optimizations to optimize codon usage in the selected host cell, humanization or tagging. The selected sequence can encode a secreted, cytoplasmic, nuclear, membrane bound or cell surface polypeptide.

The “protein of interest” includes proteins, polypeptides, fragments thereof, peptides, all of which can be expressed in the selected host cell. Desired proteins can be for example antibodies, enzymes, cytokines, lymphokines, adhesion molecules, receptors and derivatives or fragments thereof, and any other polypeptides that can serve as agonists or antagonists and/or have therapeutic or diagnostic use. Examples for a desired protein/polypeptide are also given below.

In the case of more complex molecules such as monoclonal antibodies the GOI encodes one or both of the two antibody chains.

The “product of interest” may also be an antisense RNA.

“Proteins of interest” or “desired proteins” are those mentioned above. Especially, desired proteins/polypeptides or proteins of interest are for example, but not limited to insulin, insulin-like growth factor, hGH, tPA, cytokines, such as interleukines (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF), such as TNF alpha and TNF beta, TNF gamma, TRAIL; G-CSF, GM-CSF, M-CSF, MCP-1 and VEGF. Also included is the production of erythropoietin or any other hormone growth factors. The method according to the invention can also be advantageously used for production of antibodies or fragments thereof. Such fragments include e.g. Fab fragments (Fragment antigen-binding=Fab). Fab fragments consist of the variable regions of both chains which are held together by the adjacent constant region. These may be formed by protease digestion, e.g. with papain, from conventional antibodies, but similar Fab fragments may also be produced in the mean time by genetic engineering. Further antibody fragments include F(ab′)2 fragments, which may be prepared by proteolytic cleaving with pepsin.

The protein of interest is preferably recovered from the culture medium as a secreted polypeptide, or it can be recovered from host cell lysates if expressed without a secretory signal. It is necessary to purify the protein of interest from other recombinant proteins and host cell proteins in a way that substantially homogenous preparations of the protein of interest are obtained. As a first step, cells and/or particulate cell debris are removed from the culture medium or lysate. The product of interest thereafter is purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin such as DEAE. In general, methods teaching a skilled person how to purify a protein heterologous expressed by host cells, are well known in the art.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilised. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins of this kind are well known from the art.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucin-zipper domains. However, there are also strategies in which the interaction between the VH/VL regions of the scFv are used for the multimerisation (e.g. dia-, tri- and pentabodies). By diabody the skilled person means a bivalent homodimeric scFv derivative. The shortening of the Linker in an scFv molecule to 5-10 amino acids leads to the formation of homodimers in which an inter-chain VH/VL-superimposition takes place. Diabodies may additionally be stabilised by the incorporation of disulphide bridges. Examples of diabody-antibody proteins are well know from the art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a Linker region. Examples of minibody-antibody proteins are well known from the art.

By triabody the skilled person means a: trivalent homotrimeric scFv derivative. ScFv derivatives wherein VH-VL are fused directly without a linker sequence lead to the formation of trimers.

By “scaffold proteins” a skilled person means any functional domain of a protein that is coupled by genetic cloning or by co-translational processes with another protein or part of a protein that has another function.

The skilled person will also be familiar with so-called miniantibodies which have a bi-, tri- or tetravalent structure and are derived from scFv. The multimerisation is carried out by di-, tri- or tetrameric coiled coil structures.

By definition any sequences or genes introduced into a host cell are called “heterologous sequences” or “heterologous genes” or “transgenes” with respect to the host cell, even if the introduced sequence or gene is identical to an endogenous sequence or gene in the host cell.

A “heterologous” protein is thus a protein expressed from a heterologous sequence.

The term “recombinant” is used exchangeably with the term “heterologous” throughout the specification of this present invention, especially in the context with protein expression. Thus, a “recombinant” protein is a protein expressed from a heterologous sequence.

Heterologous gene sequences can be introduced into a target cell by using an “expression vector”, preferably an eukaryotic, and even more preferably a mammalian expression vector. Methods used to construct vectors are well known to a person skilled in the art and described in various publications. In particular techniques for constructing suitable vectors, including a description of the functional components such as promoters, enhancers, termination and polyadenylation signals, selection markers, origins of replication, and splicing signals, are reviewed in considerable details in (Sambrook et al., 1989) and references cited therein. Vectors may include but are not limited to plasmid vectors, phagemids, cosmids, articificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif.

In a preferred embodiment the expression vector comprises at least one nucleic acid sequence which is a regulatory sequence necessary for transcription and translation of nucleotide sequences that encode for a peptide/polypeptide/protein of interest.

The term “expression” as used herein refers to transcription and/or translation of a heterologous nucleic acid sequence within a host cell. The level of expression of a desired product/protein of interest in a host cell may be determined on the basis of either the amount of corresponding mRNA that is present in the cell, or the amount of the desired polypeptide/protein of interest encoded by the selected sequence as in the present examples. For example, mRNA transcribed from a selected sequence can be quantitated by Northern blot hybridization, ribonuclease RNA protection, in situ hybridization to cellular RNA or by PCR. Proteins encoded by a selected sequence can be quantitated by various methods, e.g. by ELISA, by Western blotting, by radioimmunoassays, by immunoprecipitation, by assaying for the biological activity of the protein, by immunostaining of the protein followed by FACS analysis or by homogeneous time-resolved fluorescence (HTRF) assays.

“Transfection” of eukaryotic host cells with a polynucleotide or expression vector, resulting in genetically modified cells or transgenic cells, can be performed by any method well known in the art. Transfection methods include but are not limited to liposome-mediated transfection, calcium phosphate co-precipitation, electroporation, polycation (such as DEAE-dextran)-mediated transfection, protoplast fusion, viral infections and microinjection. Preferably, the transfection is a stable transfection. The transfection method that provides optimal transfection frequency and expression of the heterologous genes in the particular host cell line and type is favoured. Suitable methods can be determined by routine procedures. For stable transfectants the constructs are either integrated into the host cell's genome or an artificial chromosome/mini-chromosome or located episomally so as to be stably maintained within the host cell.

The invention relates to a method for increasing protein, preferably recombinant protein expression in a cell comprising
a. Providing a cell,
b. Increasing the amount of ribosomal RNA in said cell, and
c. Cultivating said cell under conditions which allow protein expression.

In a specific embodiment step b) comprises upregulating ribosomal RNA transcription in said host cell, preferably by reducing ribosomal RNA gene (rDNA) silencing in said cell (epigenetic engineering of at least one ribosomal RNA gene (rDNA)).

The invention specifically relates to a method for increasing protein, preferably recombinant protein expression in a cell comprising
a. Providing a cell,
b. Increasing the amount of ribosomal RNA in said cell by reducing ribosomal RNA gene (rDNA) silencing in said cell, and
c. Cultivating said cell under conditions which allow protein expression.

In a specific embodiment step b) comprises epigenetic engineering of at least one ribosomal RNA gene (rDNA).

The invention preferably relates to a method for increasing protein, preferably recombinant protein expression in a cell comprising
a. Providing a cell,
b. Reducing ribosomal RNA gene (rDNA) silencing in said cell, and
c. Cultivating said cell under conditions which allow protein expression.

In a specific embodiment of the present invention recombinant protein expression is increased in said cell compared to a cell with no reduced rDNA silencing. Preferably said increase is 20% to 100%, more preferably 20% to 300%, most preferably more than 20%. In a further specific embodiment of the present invention method step b) comprises the knock-down or knock-out of a component of the nucleolar remodelling complex (NoRC). Specifically step b) comprises reducing the expression of a component of the nucleolar remodelling complex (NoRC).

In another preferred embodiment of the present invention the NoRC component is TIP-5 or SNF 2H, preferably TIP-5.

In a very preferred embodiment of the present invention TIP-5 is knocked out.

In another embodiment of the present invention SNF2H is knocked out.

In a specific embodiment of the method of the present invention TIP-5 is knocked down or knocked out, whereby the TIP-5 silencing vector comprises:
a. shRNA according to SEQ ID NO:1, SEQ ID NO:2, SEQ ID NO:8 or SEQ ID NO:9 or
b. miRNA according to SEQ ID NO: 3, SEQ ID NO:4, SEQ ID NO:10 or SEQ ID NO:11.

In a most preferred embodiment of the present invention TIP-5 is knocked-down in step b).

The invention further relates to a method for producing a protein of interest comprising
a. Providing a cell,
b. Increasing the amount of ribosomal RNA in said cell,
c. Cultivating said cell under conditions which allow expression of said protein of interest.

In a specific embodiment of the present invention the method additionally comprises
d. Purifying said protein of interest.

In a specific embodiment the cell of step a) is a empty host cell. In another embodiment said cell of step a) is a recombinant cell comprising a gene encoding for a protein of interest.

In a further specific embodiment, step b) comprises increasing the amount of ribosomal RNA (upregulating ribosomal RNA transcription) in said cell by reducing ribosomal RNA gene (rDNA) silencing in said cell (epigenetic engineering of at least one rDNA).

The invention specifically relates to a method for producing a protein of interest comprising
a. Providing a cell,
b. Reducing ribosomal RNA gene (rDNA) silencing in said cell (epigenetic engineering of at least one rDNA), and
c. Cultivating said cell under conditions which allow expression of said protein of interest.

In a further embodiment of the present invention the method additionally comprises
d. Purifying said protein of interest.

In a specific embodiment step b) comprises the knock-down or knock-out of a component of the nucleolar remodelling complex (NoRC). In another embodiment step b) comprises reducing the expression of a component of the nucleolar remodelling complex (NoRC).

In a very preferred embodiment of the invention the NoRC component is TIP-5 or SNF 2H, most preferably TIP-5.

In a specific embodiment of the above method for producing a protein TIP-5 is knocked down or knocked out, whereby the TIP-5 silencing vector comprises:
a. shRNA according to SEQ ID NO:1, SEQ ID NO:2, SEQ ID NO:8 or SEQ ID NO:9 or
b. miRNA according to SEQ ID NO: 3, SEQ ID NO:4, SEQ ID NO:10 or SEQ ID NO:11.

The invention furthermore relates to a method of generating a host cell, preferably for production of recombinant/heterologous protein comprising
a. Providing a cell,
b. Increasing the amount of ribosomal RNA in said cell.

The invention specifically relates to a method of generating a host cell, preferably for production of recombinant/heterologous protein comprising
a. Providing a cell,
b. Increasing the amount of ribosomal RNA in said cell,
c. Obtaining a host cell.

The invention further relates to a method of generating a single cell clone, preferably for production of recombinant/heterologous protein comprising
a. Providing a cell,
b. Increasing the amount of ribosomal RNA in said cell,
c. Selecting a single cell clone.

The invention furthermore relates to a method of generating a host cell line, preferably for production of recombinant/heterologous proteins comprising
a. Providing a cell,
b. Increasing the amount of ribosomal RNA in said cell,
c. Selecting a single cell clone.

In a specific embodiment of the present invention the method additionally comprises
d. Obtaining a host cell line from said single cell clone.

The invention furthermore relates to a method of generating a monoclonal host cell line, preferably for production of recombinant/heterologous proteins comprising
a. Providing a cell,
b. Increasing the amount of ribosomal RNA in said cell,
c. Selecting a monoclonal host cell line.

In a specific embodiment of the above methods, step b) comprises increasing the amount of ribosomal RNA (upregulating ribosomal RNA transcription) in said cell by i) reducing ribosomal RNA gene (rDNA) silencing in said cell (epigenetic engineering of at least one rDNA).

The invention specifically relates to a method of generating a host cell (line), preferably for production of recombinant/heterologous proteins comprising
a. Providing a cell,
b. Reducing ribosomal RNA gene (rDNA) silencing in said cell (epigenetic engineering of at least one rDNA).

Optionally said method additionally comprises
c. Selecting a single cell clone.
d. Preferably said method additionally comprises Obtaining a host cell (line).

In a specific embodiment step b) comprises the knock-down or knock-out of a component of the nucleolar remodelling complex (NoRC). In another embodiment step b) comprises reducing the expression of a component of the nucleolar remodelling complex (NoRC).

In a very preferred embodiment of the invention the NoRC component is TIP-5 or SNF 2H, most preferably TIP-5.

In a specific embodiment of the above method of generating a host cell TIP-5 is knocked down or knocked out, whereby the TIP-5 silencing vector comprises:
a. shRNA according to SEQ ID NO:1, SEQ ID NO:2, SEQ ID NO:8 or SEQ ID NO:9 or
b. miRNA according to SEQ ID NO: 3, SEQ ID NO:4, SEQ ID NO:10 or SEQ ID NO:11.

The invention further relates to a cell generated according to any of the above methods. Preferably, the expression of recombinant protein is increased in said cell compared to a cell with no reduced rDNA silencing, preferably said increase is 20% to 100%, more preferably 20% to 300%, most preferably more than 20%.

Preferably, said cell or the cell in any of the above described methods is a eukaryotic cell, preferably a mammalian, rodent or hamster cell. Preferably, said hamster cell is a Chinese Hamster Ovary (CHO) cell such as CHO-DG44, CHO-K1, CHO-S or CHO-DUKX B11, preferably said cell is a CHO-DG44 cell.

The invention further relates to a use of said cell, preferably for the production of a protein of interest.

The invention further relates to a TIP-5 silencing vector comprising
a. shRNA according to SEQ ID NO:1, SEQ ID NO:2, SEQ ID NO:8 or SEQ ID NO:9, or
b. miRNA according to SEQ ID NO: 3, SEQ ID NO:4, SEQ ID NO:10 or SEQ ID NO:11.

Furthermore, the present invention relates to a cell comprising a TIP-5 silencing vector. Preferably such cell additionally comprises (contains) a vector containing an expression cassette comprising a gene encoding a protein of interest.

The invention further relates to a cell in which TIP-5 has been knocked out and which optionally comprises a vector including an expression cassette comprising a gene encoding a protein of interest. Preferably, said knock-out cell is a complete knock-out. In another embodiment the invention relates to a cell with deleted TIP-5 and which optionally comprises a vector including an expression cassette comprising a gene encoding a protein of interest.

The invention further relates to a kit comprising a TIP-5 silencing vector. Preferably such a kit is used for manufacturing a protein of interest. Preferably such a kit additionally comprises a cell (host cell, such as described above). Preferably such a kit comprises a TIP-5 knock-out cell as described above. Optionally said kit comprises cell culture medium and/or a transfection agent.

The practice of the present invention will employ, unless otherwise indicated, conventional techniques of cell biology, molecular biology, cell culture, immunology and the like which are in the skill of one in the art. These techniques are fully disclosed in the current literature.

### Materials and Methods

### Plasmids

pCMV-TAP-tag contains TAP-tag sequences transcribed under control of cytomegalovirus immediate early promoter.

### Stable Cell Lines

NIH/3T3 cells are stably transfected with plasmids expressing shRNA TIP5-1 (5′-GGA-CGATAAAGCAAAGATGTTCAAGAGACATCTTTGCTTTATCGTCC3′ SEQ ID NO:1) and TIP5-2 (5′-GCAGCCCAGGGAAACTAGATTCAAGAGATCTAGTTTCCC-TGGGCTGC3′ SEQ ID NO:2) sequences under control of the H1 promoter.

The transcribed shRNA sequences are: shRNA TIP5-1.1 (5′-GGACGAUAAAGCAAA-GAUGUUCAAGAGACAUCUUUGCUUUAUCGUCC3′ SEQ ID NO:8) and shRNA TIP5-2.1 (5′-GCAGCCCAGGGAAACUAGAUUCAAGAGAUCUAGUUUCCC-UGGGCUGC3′ SEQ ID NO:9)

HEK293T and CHO-K1 cells are stably transfected with plasmids expressing control miRNA or miRNA sequences targeting TIP5 (TIP5-1: 5′-GATCAG-CCGCAAACTCCTCTGAGTTTTGGCCACTGACTGACTCAGAGGATTG CGGCTGAT-3′ SEQ ID NO:3; TIP5-2: 5′-GCAAAGATGGGATCAGTTAAGGGTTTT-GGCCACTGACTGACC CTTAACTTCCCATCTTTG-3′ SEQ ID NO:4) according to the Block-iT Pol II miR RNAi system (Invitrogen). Infections were performed according to manufacture instructions. Cells were analyzed 10 days after infection.

The transcribed miRNA sequences are: miRNA TIP5-1.1: 5′-GAUCAG-CCGCAAACUCCUCUGAGUUUUGGCCACUGACUGACUCAGAGGAUUG CGGCUGAU-3′ SEQ ID NO:10; and miRNA TIP5-2.1: 5′-GCAAAGAUGGGAUCA-GUUAAGGGUUUUGGCCACUGACUGACC CUUAACUUCCCAUCUUUG-3′ SEQ ID NO:11)

### Transcription Analysis

45S pre-rRNA transcription is measured by qRT-PCR in accordance with the standard procedure and using the Universal Master mix (Diagenode). Primer sequences used to detect mouse and human 45S pre-rRNA and GAPDH have been described before.

### CpG Methylation Analysis

Methylation of mouse and human rDNA is measured as described previously. Primers used for analysis of rDNA methylation in CHO-K1 cells are: −168/−149 forward 5′-GACCAG-TTGTTGCTTTGATG-3′ SEQ ID NO:5; −10/+10 reverse 5′ GCGTGTCAGTACCTATCT-GC-3′ SEQ ID NO:6; −100/−84 forward 5′-TCCCGACTTCCAGAATTTC-3′ SEQ ID NO:7.

### BrUTP Incorporation

For BrUTP incorporation, coverslips seeded with shRNA control and TIP5-1 and 2 cells are incubated with KH buffer containing 10 mM BrUTP for 10 minutes. Then, BrUTP KH buffer is removed and the cells are incubated 30 minutes in growth medium containing 20% FCS to chase the transcripts before fixation. The cells are fixed in 100% methanol for 20 minutes at −20° C., air-dried for 5 minutes and rehydrated with PBS for 5 minutes. BrUTP incorporation is then detected using monoclonal anti-BrdU antibodies (Sigma-Aldrich).

### Growth Curves

10⁵ cells were seeded per well of a 6-well plate and each day cells were trypsinized, collected and counted with Casy® Cell Counter (Schaerfe System). Experiments are performed in duplicates and repeated twice.

### Polysome Profile

Cells are treated with cycloheximide (100 μg/ml, 10 min) and lysed in 20 mM Tris-HCl, pH7.5, 5 mM MgCl₂, 100 mM KCl, 2.5 mM DTT, 100 μg/ml cycloheximide, 0.5% NP40, 0.1 mg/ml heparin and 200 U/ml RNAse inhibitor at 4° C. After centrifugation at 8,000 g for 5 min, the supernatants are loaded onto a 15%-45% sucrose gradient and centrifuged for 4 h at 28,000 rpm at 4° C. 200 n1 fractions are collected and the optical density of individual fractions is measured at 260 nm.

### Protein Production

Protein production is assessed 48 h after transfection of a constitutive SEAP (pCAG-SEAP) or luciferase expression vector (pCMV-Luciferase). SEAP production is measured by a p-nitrophenyphospate-based light-absorbance time course. Luciferase profiling is performed according to the manufacturer's instructions (Applied biosystems, Tropix® luciferase assay kit). Values are normalized to cell numbers and to transfection efficiency. Transfection efficiency is measured by flowcytometric analysis of cells transfected with a GFP expression vector (GFP-C1, Clontech). All experiments are performed in triplicate and are repeated three times.

### Cell Culture of Suspension Cells

All cell lines used at production and development scale are maintained in serial seedstock cultures in surface-aerated T-flasks (Nunc, Denmark) in incubators (Thermo, Germany) or shake flasks (Nunc, Denmark) at a temperature of 37° C. and in an atmosphere containing 5% CO₂. Seedstock cultures are subcultivated every 2-3 days with seeding densities of 1-3E5 cells/mL. The cell concentration is determined in all cultures by using a hemocytometer. Viability is assessed by the trypan blue exclusion method.

### Fed-Batch Cultivation

Cells are seeded at 3E05 cells/ml into 125 ml shake flasks in 30 ml of BI-proprietary production medium without antibiotics or MTX (Sigma-Aldrich, Germany). The cultures are agitated at 120 rpm in 37° C. and 5% CO₂ which is reduced to 2% following day 3. BI-proprietary feed solution is added daily and pH is adjusted to pH 7.0 using NaCO₃ as needed. Cell densities and viability are determined by trypan-blue exclusion using an automated CEDEX cell quantification system (Innovatis).

### Generation of Antibody-Producing Cells

CHO-K1 or CHO-DG44 cells (Urlaub et al., Cell 1983) are stably transfected with expression plasmids encoding heavy and light chains of an IgG1-type antibody. Selection is carried out by cultivation of transfected cells in the presence of the respective antibiotics encoded by the expression plasmids. After about 3 weeks of selection, stable cell populations are obtained and further cultivated according to a standard stock culture regime with subcultivation every 2 to 3 days. In a next (optional) step, FACS-based single cell cloning of the stably transfected cell populations is carried out to generate monoclonal cell lines.

### Determination of Recombinant Antibody Concentration

To assess recombinant antibody production in transfected cells, samples from cell supernatant are collected from standard inoculum cultures at the end of each passage for three consecutive passages. The product concentration is then analysed by enzyme linked immunosorbent assay (ELISA). The concentration of secreted monoclonal antibody product is measured using antibodies against human-Fc fragment (Jackson Immuno Research Laboratories) and human kappa light chain HRP conjugated (Sigma).

### EXAMPLES

### Example 1

### Knock-Down of TIP-5

With the aim of engineering cells for increased synthesis of recombinant proteins, we determine whether a decrease in the number of silent rRNA genes enhances 45S pre-rRNA synthesis and, as consequence, also stimulates ribosome biogenesis and increases the number of translation-competent ribosomes. Therefore, we use RNA interference to knock down TIP5 expression and constructed stably transgenic shRNAexpressing NIH/3T3 or miRNA-expressing HEK293T and CHO-K1 using shRNA/miRNA sequences specific for two different regions of TIP5 (TIP5-1 and TIP5-2). Stable cell lines expressing scrambled shRNA and miRNA sequences were used as control. There are two reasons for producing stable cell lines rather than performing transient transfections with plasmids expressing shRNA-TIP5 or miRNA-TIP5 sequences. First, the loss of repressive epigenetic marks like CpG methylation is a passive mechanism, requiring multiple cell divisions. Second, even though HEK293T cells can be transfected relatively easily, the poor transfection efficiency of NIH/3T3 and CHO-K1 cells would compromise subsequent analyses of endogenous rRNA, ribosome levels and cell growth properties. To determine the efficiency of TIP5 knockdown in the selected clones, we measure TIP5 mRNA levels by quantitative and semiquantitative reverse-transcriptase-mediated PCR (FIG. 1). TIP5 expression decreases about 70-80% in NIH/3T3/shRNA-TIP5-1 and -2 cells when compared to control cells (FIG. 1A). A similar reduction in TIP5 mRNA levels is observed in stable HEK293T (FIG. 1B). TIP5 mRNA levels in CHO-K1-derived cells could be measured only by semiquantitative PCR (FIG. 1C) but the reduction of TIP5 mRNA was similar to that of stable NIH/3T3 and HEK293T cells. These results demonstrate that the established cell lines contain low levels of TIP5.

### Example 2

### TIP-5 Knockdown Leads to Reduced rDNA Methylation

In NIH/3T3 cells about 40% to 50% of rRNA genes contain CpG-methylated sequences and are transcriptionally silent. The sequences and CpG density of the rDNA promoter in humans, mice and Chinese hamsters differ significantly. In humans, the rDNA promoter contains 23 CpGs, while in mice and Chinese hamsters there are 3 and 8 CpGs, respectively (FIG. 2A-C). To verify that TIP5 knockdown affects rDNA silencing, we determine the rDNA methylation levels by measuring the amount of meCpGs in the CCGG sequences. Genomic DNA is HpaII-digested, and resistance to digestion (i.e. CpG methylation) is measured by quantitative real-time PCR using primers encompassing HpaII sequences (CCGG). There is a decrease in CpG methylation within the promoter region of a the majority of rRNA genes in all TIP5 knock-down cell lines, underscoring the key role of TIP5 in promoting rDNA silencing (FIG. 2).

Notably, although TIP5 binding and de novo methylation is restricted to the rDNA promoter sequences, CpG methylation amounts in TIP-5 reduced NIH3T3 cells diminished over the entire rDNA gene (intergenic, promoter and coding regions; FIG. 2D,E), indicating that TIP5, once bound to the rDNA promoter, initiates spreading mechanisms for the establishment of silent epigenetic marks throughout the rDNA locus.

### Example 3

### Increased rRNA Levels in TIP-5 Knockdown Cells

To determine whether a decrease in the number of silent genes affects the amounts of the rRNA transcript, we measure 45S pre-rRNA synthesis by qRT-PCR using primers that encompassed the first rRNA processing site (FIG. 3A) and by in vivo BrUTP incorporation (FIG. 3B). As expected, in both TIP5-depleted NIH/3T3 and HEK293T cells, an enhancement of rRNA production compared to the control cell line is detected by both analyses

### Example 4

### TIP-5 Depletion Leads to Increased Proliferation and Cell Growth

Ras is a well known oncogene involved in cell transformation and tumorigenesis which is frequently mutated or overexpressed in human cancers. Green et al., 2009; WO2009/017670 describe to have identified TIP-5 to function as a Ras-mediated epigenetic silencing effector (RESE) of Fas in a global miRNA screen. The publication describes that reduced expression of Ras effectors such as TIP-5 results in an inhibition of cell proliferation.

We analyze both shRNA-TIP5 cells by flow cytometry (FACS). As shown in FIGS. 4A,B, the numbers of cells in S-phase were significantly higher in both shRNA-TIP5 cells in comparison to control cells. A similar profile was obtained with NIH3T3 cells 10 days after infection with a retrovirus expressing miRNA directed against TIP5 sequences. Consistent with these results, shRNA TIP5 cells show increased incorporation of 5-bromodeoxyuridine (BrdU) into nascent DNA and higher levels of Cyclin A (FIG. 4C). Finally, we compare cell proliferation rates between shRNA-TIP5, shRNA-control and parental NIH3T3, HEK293 and CHO-K1 cells (FIG. 4D-F). Surprisingly and in contrast to the prior art reports, both NIH/3T3 and CHO-K1 cells, expressing miRNA-TIP5 sequences, proliferate at faster rates than the control cells, suggesting that a decrease in the number of silent rRNA genes does have an impact on cell metabolism. TIP5 depletion in HEK293T did not significantly affect cell proliferation, because these cells had already reached their maximum rate of proliferation. These data surprisingly show that depletion of TIP5 and a consequent decrease in rDNA silencing enhance cell proliferation.

### Example 5

### Ribosome Analysis in TIP-5 Knockdown Cells

In mammalian cell cultures, the rate of protein synthesis is an important parameter, which is directly related to the product yield. To determine whether depletion of TIP5 and a consequent decrease in rDNA silencing increases the number of translation-competent ribosomes in the cell, we initially measure the levels of cytoplasmic rRNA. In the cytoplasm, most of the RNA consists of processed rRNAs assembled into ribosomes. As shown in FIG. 5A-C, all TIP5-depleted cell lines containe more cytoplasmic RNA per cell, suggesting that these cells produce more ribosomes. Also, analysis of the polysome profile shows that TIP5depleted HEK293 and CHO-K1 cells contained more ribosome subunits (40S, 60S and 80S) compared to control cells (FIG. 5D).

### Example 6

### TIP-5 Knockdown Leads to Enhanced Production of Reporter Proteins

To determine whether depletion of TIP5 and decrease in rDNA silencing enhance heterologous protein production, we transfect stable TIP5-depleted NIH/3T3, HEK293T and CHO-K1 derivatives with expression vector promoting constitutive expression of the human placental secreted alkaline phosphatase SEAP (pCAG-SEAP; FIG. 6A-C) or luciferase (pCMV-luciferase; (FIG. 6D,E). Quantification of protein production after 48 h reveals a two- to four-fold increase in both SEAP and luciferase production in TIP5-depleted cells compared to the control cell lines, indicating that TIP5-depletion increases heterologous protein production. All these results show that a decrease in the number of silent rRNA genes enhances ribosome synthesis and increases the potential of the cells to produce recombinant proteins.

### Example 7

### TIP-5 Knockout Increases Biopharmaceutical Production of Monocyte Chemoattractant Protein 1 (MCP-1)

(a) A CHO cell line (CHO DG44) secreting monocyte chemoattractant protein 1 (MCP-1) is transfected with an empty vector (MOCK control) or small RNAs (shRNA or RNAi) designed to knock-down TIP-5 expression. The cells are subsequently subjected to selection to obtain stable cell pools. During six subsequent passages, supernatant is taken from seed-stock cultures of both, mock and TIP-5 depleted stable cell pools, the MCP-1 titer is determined by ELISA and divided by the mean number of cells to calculate the specific productivity. The highest MCP-1 titers are seen in the cell pools with the most efficient TIP-5 depletion, whereas the protein concentrations are markedly lower in mock transfected cells or the parental cell line.
b) CHO host cells (CHO DG44) are first transfected with short RNAs sequences (shRNAs or RNAi) to reduce TIP-5 expression and stable TIP-5 depleted host cell lines are generated. Subsequently these cell lines and in parallel CHO DG 44 wild type cells are transfected with a vector encoding monocyte chemoattractant protein 1 (MCP-1) as the gene of interest. After a second round of selection, supernatant is taken from seed-stock cultures of all stable cell pools over a period of four subsequent passages, the MCP-1 titer is determined by ELISA and divided by the mean number of cells to calculate the specific productivity. The highest MCP-1 titers and productivities are seen in the cell pools with the most efficient TIP-5 depletion, whereas the protein concentrations are markedly lower in mock transfected cells or the parental cell line.
c) When the same cells described in a) or b) are subjected to batch or fed-batch fermentations, the differences in overall MCP-1 titers are even more pronounced: As the cells transfected with reduced expression of TIP-5 grow faster and also produce more protein per cell and time, they exhibit higher IVCs and show higher productivities at the same time. Both properties have a positive influence on the overall process yield. Therefore, Tip5 deleted cells have significantly higher MCP-1 harvest titers and lead to more efficient production processes.

### Example 8

### Knock-Out of the TIP-5 Gene Increases rRNA Transcription and Enhances Proliferation Most Efficiently

The most efficient way to generate an improved production host cell line with constantly reduced levels of TIP-5 expression is to generate a complete knock-out of the TIP-5 gene. For this purpose, one can either use homologous recombination or make use of the Zink-Finger Nuclease (ZFN) technology to disrupt the Tip-5 gene and prevent its expression. As homologous recombination is not efficient in CHO cells, we design a ZFN which introduces a double strand break within the TIP-5 gene which is thereby functionally destroyed. To control efficient knock-out of TIP-5, a Western Blot is performed using anti-TIP-5 antibodies. On the membrane, no TIP-5 expression is detected in TIP-5 knock-out cells wherease the parental CHO cell line shows a clear signal corresponding to the TIP-5 protein.

Next, rRNA transcription is analysed in TIP-5 knock-out CHO cells and the parental CHO cell line. The assay confirms higher levels of rRNA synthesis and increased ribosome numbers in TIP-5 knock-out cells compared to either the parental cell and also compared to cells with only reduced TIP-5 expression levels.

Moreover, cells deficient for TIP-5 proliferate faster and show higher cell numbers in fed-batch processes compared to TIP5 wild-type cells and cell lines in which TIP-5 expression was only reduced by introduction of interfering RNAs (such as shRNA or RNAi).

### Example 9

### Enhanced Therapeutic Antibody Production in TIP-5 Depleted Cells

(a) A CHO cell line (CHO DG44) secreting a human monoclonal IgG subtype antibody is transfected with an empty vector (MOCK control) or small RNAs (shRNA or RNAi) designed to knock-down TIP-5 expression. The cells are subsequently subjected to selection to obtain stable cell pools. Alternatively, TIP-5 is depleted by deletion of the TIP-5 gene (knock-out). During six subsequent passages, supernatant is taken from seed-stock cultures of both, mock and TIP-5 depleted stable cell pools, antibody titers are determined by ELISA and divided by the mean number of cells to calculate the specific productivity. The highest IgG titers are measured in the cultures of TIP-5 depleted cells, whereas the protein concentrations are markedly lower in mock transfected cells or the parental cell line.
b) TIP-5 is depleted in CHO host cells (CHO DG44) either by transfection with short RNAs sequences (shRNAs or RNAi) hybridizing to TIP-5 sequences or by stable knock-out of the TIP-5 gene. Subsequently these cell lines and in parallel CHO DG 44 wild type cells are transfected with expression constructs encoding heavy and light chains of an antibody as the gene of interest. Stably transfected cell populations are generated and supernatant is taken from seed-stock cultures of all stable cell pools over a period of four subsequent passages. The antibody concentrations in the culture supernatants are determined by ELISA and divided by the mean number of cells to calculate the specific productivity. Cell pools derived from TIP-5 depleted cells show the highest antibody titers and productivities compared to MOCK controls and the parental unmodified DG44 cell line which produce markedly lower IgG amounts.
c) When the same cells described in a) or b) are subjected to batch or fed-batch fermentations, the differences in overall antibody titers are even more pronounced: As the TIP-5 depleted cells grow faster and also produce more protein per cell and time, they exhibit higher IVCs and show higher productivities at the same time. Both properties have a positive influence on the overall process yield. Therefore, Tip5 deleted cells have significantly higher IgG harvest titers and lead to more efficient production processes.

### Example 10

### Knock-Down of SNF2H Leads to Increased Protein Production and Improved Cell Growth

(a) A CHO cell line (CHO DG44) secreting a human monoclonal IgG subtype antibody is transfected with an empty vector (MOCK control) or small RNAs (shRNA or RNAi) designed to knock-down SNF2H expression. The cells are subsequently subjected to selection to obtain stable cell pools. Alternatively, SNF2H is depleted by deletion/disruption of the SNF2H gene (knock-out). During six subsequent passages, supernatant is taken from seed-stock cultures of both, mock and SNF2H depleted stable cell pools, antibody titers are determined by ELISA and divided by the mean number of cells to calculate the specific productivity. The highest IgG titers are measured in the cultures of SNF2H depleted cells, whereas the protein concentrations are markedly lower in mock transfected cells or the parental cell line.
b) SNF2H is depleted in CHO host cells (CHO DG44) either by transfection with short RNAs sequences (shRNAs or RNAi) hybridizing to SNF2H sequences or by knock-out of the SNF2H gene. Subsequently these cell lines and in parallel CHO DG 44 wild type cells are transfected with expression constructs encoding heavy and light chains of an antibody as the protein of interest. Stably transfected cell populations are generated and supernatant is taken from seed-stock cultures of all stable cell pools over a period of four subsequent passages. The antibody concentrations in the culture supernatants are determined by ELISA and divided by the mean number of cells to calculate the specific productivity. Cell pools derived from SNF2H depleted cells show the highest antibody titers and productivities compared to MOCK controls and the parental unmodified DG44 cell line which produce markedly lower IgG amounts.
c) When the same cells described in a) or b) are subjected to batch or fed-batch fermentations, the differences in overall antibody titers are even more pronounced: As the SNF2H depleted cells grow faster and also produce more protein per cell and time, they exhibit higher IVCs and show higher productivities at the same time. Both properties have a positive influence on the overall process yield. Therefore, SNF2H deleted cells have significantly higher IgG harvest titers and lead to more efficient production processes.

### SEQUENCE TABLE

### RNAs Used for TIP-5 Depletion in NIH3T3 Cells:

| | |
|---|---|
| SEQ ID NO: 1 | shRNA TIP5-1 |
| SEQ ID NO: 2 | shRNA TIP5-2 |

### RNAs Used for TIP-5 Depletion in Human and Hamster Cell Lines:

| | |
|---|---|
| SEQ ID NO: 3 | miRNA TIP5-1 |
| SEQ ID NO: 4 | miRNA TIP5-2 |

### Primers Used for Methylation Analysis

| | |
|---|---|
| SEQ ID NO: 5 | Primer −168/−149 forward |
| SEQ ID NO: 6 | Primer −10/+ reverse |
| SEQ ID NO: 7 | Primer −100/−84 forward |

### Transcribed RNA Sequences:

| | |
|---|---|
| SEQ ID NO: 8 | shRNATIP5-1.1 |
| SEQ ID NO: 9 | shRNATIP5-2.1 |
| SEQ ID NO: 10 | miRNATIP5-1.1 |
| SEQ ID NO: 11 | miRNA TIPS-2.1 |

### Genes/Proteins Described in the Present Invention:

| Protein | Official Symbol | GeneID | Human Reference Sequence |
|---|---|---|---|
| TIP-5 | BAZ2A | 11176 | NP_038477.2 |
| SNF2H | SMARCA5 | 8467 | NP_003592.2 |

1. A method for increasing recombinant protein expression in a cell comprising:
a. Providing a cell,
b. Reducing ribosomal RNA gene (rDNA) silencing in said cell, and
c. Cultivating said cell under conditions which allow protein expression,
whereby step b) comprises the knock-down or knock-out of TIP-5 or SNF 2H, wherein recombinant protein expression is increased in said cell compared to a cell with no reduced rDNA silencing.

2. The method according to claim 1, wherein recombinant protein expression is increased in said cell compared to a cell with no reduced rDNA silencing by more than 20%.

3. (canceled)

4. The method according to claim 1, whereby step b) comprises the knock-down or knock-out of TIP 5.

5. The method according to claim 1, whereby TIP-5 is knocked out.

6. The method according to claim 4, whereby said cell comprises a TIP-5 silencing vector, whereby the TIP-5 silencing vector comprises:
a. shRNA according to SEQ ID NO:1, SEQ ID NO:2, SEQ ID NO:8 or SEQ ID NO:9, or
b. miRNA according to SEQ ID NO: 3, SEQ ID NO:4, SEQ ID NO:10 or SEQ ID NO:11.

7. The method according to claim 1, whereby SNF2H is knocked out.

8. A method for producing a protein of interest in a cell comprising:
a. Providing a cell,
b. Reducing ribosomal RNA gene (rDNA) silencing in said cell,
c. Cultivating said cell under conditions which allow expression of said protein of interest, whereby said protein of interest is expressed in said cell,
whereby step b) comprises the knock-down or knock-out of TIP-5 or SNF 2H.

9. The method according to claim 8, whereby the method additionally comprises:
d. Purifying said protein of interest.

10. (canceled)

11. The method according to claim 8, whereby step b) comprises the knock-down or knock-out of TIP 5.

12. A method of generating a host cell for production of recombinant protein comprising:
a. Providing a cell,
b. Reducing ribosomal RNA gene (rDNA) silencing in said cell,
c. Optionally selecting a single cell clone,
d. Obtaining a host cell,
whereby step b) comprises the knock-down or knock-out of TIP-5 or SNF 2H.

13. (canceled)

14. The method according to claim 12, whereby step b) comprises the knock-down or knock-out of TIP-.

15. A cell generated according to the method of claim 12.

16. The cell according to claim 15, whereby the cell is a Chinese Hamster Ovary (CHO) cell, preferably aCHO-DG44, CHO-K1, CHO-S or CHO-DUKX B11, most preferably the cell is a CHO-DG44 cell.

17. A TIP-5 silencing vector comprising:
a. shRNA according to SEQ ID NO:1, SEQ ID NO:2, SEQ ID NO:8 or SEQ ID NO:9, or
b. miRNA according to SEQ ID NO: 3, SEQ ID NO:4, SEQ ID NO:10 or SEQ ID NO:11.

18. A cell comprising a TIP-5 silencing vector according to claim 16 and optionally a vector containing an expression cassette comprising a gene encoding a protein of interest.

19. A cell in which TIP-5 has been knocked out and which optionally comprises a vector including an expression cassette comprising a gene encoding a protein of interest.

20. The method according to claim 2, wherein recombinant protein expression is increased in said cell compared to a cell with no reduced rDNA silencing by 20% to 300%.

21. The method according to claim 2, wherein recombinant protein expression is increased in said cell compared to a cell with no reduced rDNA silencing by 20% to 100%.
